(19) United States
(12) Patent Application Publication
(10) Pub. No.: US 2017/0211119 A1
(43) Pub. Date: Jul. 27, 2017

(54) IN VIVO PRODUCTION OF A RECOMBINANT CAROTENOID-PROTEIN COMPLEX

(71) Applicants: Phycosource, Cergy (FR); Commissariat A L'Enerigie Atomique et Aux Energies Alteratives, Paris (FR); Centre National de la Recherche Scientifique (CNRS), Paris (FR)

(72) Inventors: Céline Bourcier de Carbon, Neuilly-sur-siene (FR); Diana Kirilovsky, Paris (FR); Clémence Boulay, Mariel-sur-Mauldre (FR); Bertrand-Elie Duran, Pantin (FR); Alexandre Phulpin, Montmorency (FR); Adjélé Wilson, Aulnay Sous Bois (FR)

(73) Assignees: Phycosource, Cergy (FR); Commissariat A L'Energie Atomique et Aux Energies Alternatives, Paris (FR); Centre National de la Recherche Scientifique, Paris (FR)

(21) Appl. No.: 15/328,949

(22) PCT Filed: Jul. 24, 2015
(86) PCT No.: PCT/EP2015/067071

§ 371 Date: Jan. 25, 2017

(30) Foreign Application Priority Data
Jul. 25, 2014 (EP) 14306207.3

(51) Int. Cl. C12P 23/00; C07K 14/195

(52) U.S. Cl. CPC C12P 23/00; C07K 14/195

(57) ABSTRACT

The present invention relates to a method for producing a carotenoid-protein complex in vivo comprising the steps of transforming of prokaryote cell with genes involved in carotenoid synthesis and with a gene encoding an apo-carotenoprotein; culturing said prokaryote cells such as to induce sequential genes expression, isolating and purifying the carotenoid-protein complex.

The present invention relates to the field of recombinant proteins.

Especially, the present application relates to a method for producing in vivo a carotenoid-protein complex and to the carotenoid-protein complex such obtained.

The present invention further relates to a modified apo-carotenoproteine genes encoding a modified carotenoid-protein complex.

The human body is constantly exposed to external harmful factors such as ultraviolet radiation, pollution, cigarette smoke, toxic chemicals, and certain metallic ions. Many of these pollutants induce the formation of reactive oxygen species (ROS) to some extent. In addition to exogenous factors, internal factors also contribute to the formation of ROS. Metabolic reactions in all living cells are accompanied by the oxidation of nutrients while reducing oxygen to water molecules.

The formation of reactive oxygen species (ROS) and the damage caused by their potent chemical activity on their surrounding environment is ubiquitous in nearly all physiological settings. Incomplete reduction of the oxygen molecule (³O₂, the ground state of the molecule) results in the formation of ROS, including the superoxide anion (O₂^{.-}, hydroperoxyl radical (HOO.), hydroxylradical (.OH), nitric oxide (.NO), and compounds that are not free radicals but have a big reactivity such as singlet oxygen (¹O₂) and hydrogen peroxide (H₂O₂).

Physiological damage induced by ROS includes oxidation of lipids, proteins and DNA.

Due to the nocive effects of ROS, there is a big interest in the production of new antioxidant molecules having better efficiency than the antioxidants currently known in the art.

In photosynthetic organisms, carotenoids have a photoprotective role through a number of different mechanisms. By quenching chlorophyll triplet states, they prevent the energy transfer-mediated formation of singlet oxygen. Chlorophyll triplets are formed with a low but significant yield, during excitation energy transfer in light-harvesting proteins and/or after charge recombination in reaction centers. Carotenoids can additionally directly quench singlet oxygen. More recently it was shown that, in both plants and cyanobacteria, carotenoids play an essential role in regulating the amount of excitation energy reaching the reaction centers in high light environments, thus preventing damage due to overexcitation of these proteins.

For majority of applications in need of anti-oxidants, the anti-oxidant effect is expected in a water-based medium. Thus, a hydrophilic antioxidant is required. However, the most efficient antioxidant, such as carotenoids are strongly hydrophobic compounds. Current hydrophilic antioxidant, such as ascorbic acid, tocopherol or citric acid are 100 to 1000 fold less protective than common carotenoid (Yasuhiro et al., 2007), so that the way to use carotenoids and other lipophilic antioxidant in aqueous environment has been long time studied.

The most common way to provide such lipophilic antioxidants requires an additional step, such as performing a hydrocolloid emulsion or saponification and micronization (WO 2009022034) or derivatization with hydrophilic function. This additional step increases the cost of methods providing efficient lipophilic antioxidant in aqueous environment.

Furthermore, performing such additional step usually leads to a decrease of antioxidant efficiency (especially related to the lack of disponibility of the antioxidant in suspension) and is not suitable for all applications due to the introduction of new compounds necessary to perform the additional step.

Thus, there remains a need to provide efficient, rapid and inexpensive methods for production of a stable, water soluble and efficient anti-oxidant compound.

Carotenoids are highly hydrophobic. Immersed in the hydrophobic core of the lipid bilayer the carotenoid is solvent protected by the amphiphilic nature of the phospholipids. In photosynthetic organisms, in addition to be solubilized in the membranes, the carotenoids are mostly associated to membrane chlorophyll proteins.

However, there also exist water soluble carotenoid binding proteins. Only a small number of proteins of this family was described and characterized till now days: for ex: the cyanobacterial “Orange Carotenoid Protein” (OCP) ((Kerfeld et al., 2003)) binding 3′-hydroxiechinenone and the cyanobacterial “Red Carotenoid Protein” (RCP) (Sedoud et al., 2014); an Astaxanthin carotenoid protein (AstaP) isolated from an eukaryotic microalgae (Kawasaki et al., 2013).

In human, when carotenoids are transported through the plasma, they are incorporated into lipoproteins (lipocalins). Carotenoids can also assemble with lipo(glycol) proteins. Finally, there exist a pi isoform of the glutathione-S-transferase (GSTP1) specifically binding zeaxanthin (Bhosale and Bernstein, 2005). Although there are very few well characterized examples of carotenoproteins, they are likely to be much more abundant in nature and can have important roles as protectants against ROS.

Crustacyanin, present in the lobster's carapace is one example of the family of astaxanthin or echinenone carotenoprotein family common in invertebrate marine animals (Zagalsky et al., 1990).

To produce these carotenoproteins, the scientists over-express only the apo-carotenoprotein (protein without carotenoid) in prokaryote cell such as E. coli and then once the apo-protein has been isolated, the carotenoid is attached by in vitro reconstitution (examples in Giuffra et al., 1996; Bhosale and Bernstein, 2005).

Alternatively they isolate them from the original organisms, for example OCP and AstaP (Wilson et al., 2008; Kawasaki et al., 2013), or from Synechocystis mutants overexpressing these proteins, for example OCP and RCP (Sedoud et al., 2014). The inventors have tested the OCP yield obtained by others conventional methods well known in the art (Bourcier de Carbon et al., 2015) and the obtained results have shown that only 40 mg of OCP have been obtained from 30 liter of Synechocystis culture (1.33 mg per liter) after 3 weeks (2 weeks for growth cells and 4 days for the isolation of the protein).

Not only such standard step methods are long but also the obtained carotenoproteins in the case of reconstitution in vitro are not always completely reconstituted or functional.

Moreover, there reminds need to provide a soluble proteins (apo-carotenoproteins) able to attach the lipid soluble carotenoid with high efficiency in order to obtain very high quantity of carotenoproteins.

The purpose of the invention is to fulfill the needs of methods allowing the production of a functional carotenoid-protein complex in high quantity which makes it possible to solve in whole or part the above mentioned problems.

Unexpectedly, the inventors have demonstrated that to obtain a large quantity of functional carotenoproteins in vivo in the prokaryotic cells it is necessary to first inducing the expression of genes encoding the carotenoids and then, inducing the expression of genes encoding apo-carotenoprotein. It is also necessary to maintain the expression of genes encoding carotenoids during the expression of genes encoding the apo-protein.

The inventors have demonstrated that when the said sequential gene expression is not observed (e.g when the expression of genes encoding the carotenoids and of genes encoding the apo-proteins starts at the same time or when the expression of genes encoding the carotenoids is decreased through the expression of the gene encoding the apo-protein) the carotenoid attachment to the apo-protein decreases and the yield of the carotenoid-protein complex (holo-protein) decreases too (cf. the comparative examples in the present application)

Thus, the major advantage of the present invention lies in a manner to transform prokaryotic cells to be able to synthesize the apo-carotenoproteins with an attached carotenoid in high quantities.

These results are surprising since contrary to the prior art methods carotenoid is attached to apo-carotenoprotein in host cell. In fact, this could be even more complicated with soluble carotenoid proteins since the isolated carotenoids are lipid-soluble and present only in membranes.

Therefore, the invention describes a biomimetic way to obtain the full anti-oxidant effect of carotenoid in aqueous environment by using carotenoid-coupled proteins such as “natural carriers”.

In one aspect the invention relates to a method for producing a carotenoid-protein complex in vivo, comprising the steps of:
a) transforming of prokaryote cells with genes involved in carotenoid synthesis and a gene encoding an apo-carotenoprotein;
b) culturing of transformed prokaryote cells in step a) in conditions allowing sequential gene expression, wherein the expression of the genes involved in carotenoid synthesis is induced prior to the expression of the gene encoding an apo-protein
c) isolating and purifying the protein-carotenoid complex expressed by the prokaryote cells.

Unless defined otherwise, all technical scientific terms used herein have the same meaning as commonly understood to one skilled in the art.

For convenience, the meaning of certain terms and phrases employed in the specification, examples and claims are provide.

As used herein the terms “carotenoprotein” or “carotenoid-protein complex” refer to a protein attached to carotenoid.

As used herein the term “prokaryote cell” refers to prokaryote cells have not a membrane bound nucleus, mitochondria, or any other membrane-bound organelles.

According to the present invention the prokaryote cells are selected in the group of non-photosynthetic prokaryote cells, preferably comprising E. coli or Lactococcus lactis.

According to the present invention E. coli strains BL21-Gold bought from Agilent Technologies (Santa Clara, Calif. 95051-7201, USA) are used as host to construct E. coli strains synthesizing carotenoids. Preferably these strains are: BL21-pβcarotene, BL21-pβcarotene, BL21echi, BL21echiBIS, BL21zea and BL21cantha (table 6 in the Examples).

As used herein the term “plasmid” or “vector” refers to a small, circular, double-stranded DNA molecule that is distinct from a cell's chromosomal DNA and that occurs in many bacterial strains.

Any one of plasmids known by the skilled in the art may be used in the present application.

Preferably the plasmids used in the present invention, their characteristics and their sources are shown in the table 4 in the Examples.

According to the method of the present invention the prokaryote cells are transformed with the genes involved in carotenoid synthesis and a gene encoding an apo-carotenoprotein.

According to one embodiment of the method of the present invention the prokaryote cells are transformed with three plasmids two of them containing the genes involved in carotenoid synthesis and the third containing a gene encoding an apo-carotenoprotein.

According to a preferred embodiment of the method of the present invention the prokaryote cells are transformed with two plasmids, one containing the genes involved in carotenoid synthesis and one containing a gene encoding an apo-carotenoprotein.

According to the most preferred embodiment of the method of the present invention the prokaryote cells are transformed with one plasmid containing the genes involved in carotenoid synthesis and containing a gene encoding an apo-carotenoprotein.

According to the present invention, genes encoding enzymes involved in carotenoid synthesis may be obtained from any organism selected in the group of any bacteria, any algae, any plant and any animal.

Preferably, the genes encoding carotenoids synthesis may be obtained from all algae or all eubacteria comprising bacteriochlorophyll photosynthetic bacteria, cyanobacteria and non-photosynthetic eubacteria. More preferably these genes are obtained from cyanobacteria strains selected in the group of Synechocystis sp., Anabaena sp. and Arthrospira sp. or from non-photosynthetic eubacteria, selected in the group of Erwinia sp, Brevundinomonas sp SD212, Paracoccus sp, and more preferably from Erwinia sp.

According to the particular embodiment, the genes involved in the β-carotene synthesis crtE, crtB, crtl and crtY are obtained from Erwinia.

Preferably, the gene involved in the β-carotene synthesis corresponds to the sequence of the Ctr operon containing crtE, crtB, crtl and crtY genes from Erwinia uredovora (SEQ ID NO: 70) or from Erwinia herbicola (SEQ ID NO: 75). The expression of these genes is preferably under the control of crtE promoter.

According to another embodiment of the present invention the prokaryote cells transformed with the plasmid containing the genes involved in β-carotene synthesis may be transformed with a second plasmid containing the genes encoding enzymes involved in the synthesis of others carotenoids and one plasmid containing a gene encoding an apo-carotenoprotein.

Preferably, the prokaryote cells transformed with the plasmid containing the genes involved in β-carotene synthesis may be transformed with a second plasmid containing the genes encoding a β-carotene ketolase or the genes encoding a β-carotene hydrolase.

More preferably, the prokaryote cells are transformed with one plasmid containing the genes involved in β-carotene synthesis and containing the genes encoding a β-carotene ketolase and/or the genes encoding a β-carotene hydrolase and one plasmid containing a gene encoding an apo-carotenoprotein.

The genes encoding a β-carotene ketolase or the genes encoding a β-carotene hydrolase are involved in the synthesis of all xanthophylls (carotenoids containing at least one oxygen atom). Preferably, said genes are selected in the group comprising the genes involved in the synthesis of echinenone, canthaxanthin, zeaxanthin, astaxanthin, hydroxyechinenone, neoxanthin, violaxanthin, diadinoxanthin and fucoxanthin. More preferably, said genes are selected in the group comprising the genes involved in the synthesis of echinenone, hydroxyechinenone, canthaxanthin, zeaxanthin and astaxanthin.

According to the invention, the genes encoding β-carotene ketolase are selected in the group comprising or consisting in crtO and crtW genes isolated from all algae or eubacteria, preferably from cyanobacteria, more preferably from cyanobacteria strains selected in the group comprising or consisting of Synechocystis sp., Anabaena sp. or Arthrospira sp., Thermosynechococcus elongatus, Synechococcus sp, and Gloeobacter sp

Still more preferably, the genes encoding a β-carotene ketolase are CrtO from Synechocystis (SEQ ID NO: 5) and CrtW from Anabaena (SEQ ID NO: 7) which are necessary for the synthesis of echinenone and canthaxanthine respectively.

According to the invention, the genes encoding β-carotene hydrolase are selected in the group comprising or consisting of crtZ from eubacteria (SEQ ID NO: 69) and crtR from all eubacteria, algae or cyanobacteria strains, preferably selected in the group of cyanobacterial strains Synechocystis sp., Anabaena sp. or Arthrospira sp., Thermosynechococcus elongatus, and Synechococcus sp

More preferably, the gene encoding a β-carotene hydrolase is CrtR from Synechocystis (SEQ ID NO: 6) which is necessary for the synthesis of zeaxanthin.

According to the present application the sequences of primers used for cloning the genes encoding carotenoids may be synthesized by any method well known to person skilled in the art.

Preferably the sequences of primers used for cloning the genes encoding carotenoid Crt genes correspond to SEQ ID NO: 19 to SEQ ID NO: 28 and SEQ ID NO: 71 and 72.

According to the method of the present invention the plasmid containing the genes involved in β-carotene synthesis further contains the genes encoding a β-carotene-ketolase and/or a β-carotene-hydrolase. The gene expression of ketolase and hydrolase is performed under the control of inducible promoter, preferably ara promoter.

According to the method of the present invention the prokaryote cells are also transformed with a plasmid containing a apo-carotenoprotein genes.

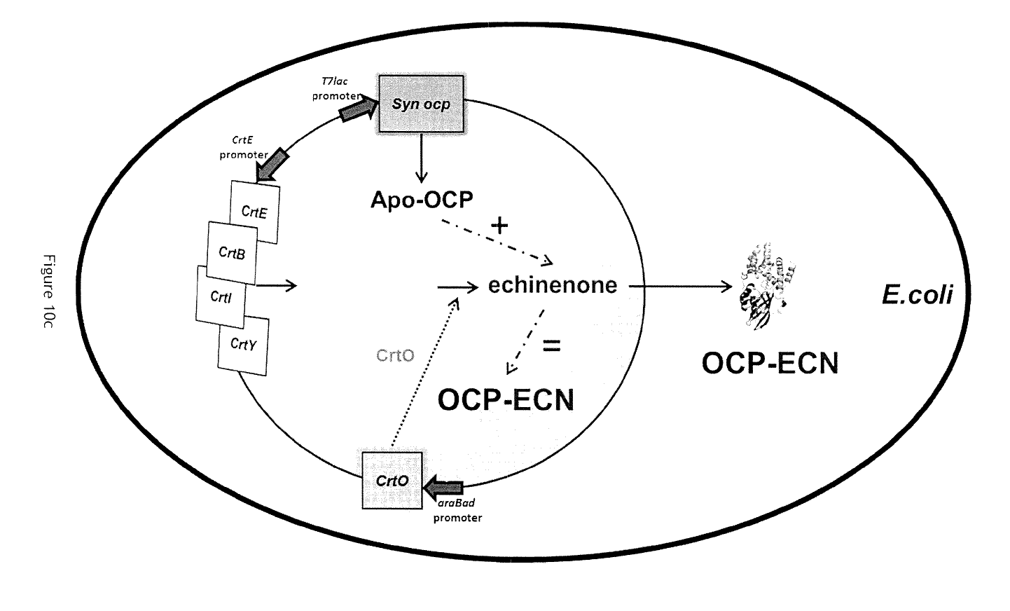

According to one particular embodiment of the method of the invention, the prokaryote cells are transformed with three plasmids: a plasmid containing the genes involved in β-carotene synthesis, a plasmid containing the genes encoding a β-carotene-ketolase and/or a β-carotene-hydrolase and a plasmid containing the genes encoding an apo-carotenoprotein (FIG. 10a).

Preferably, the prokaryote cells are transformed with two plasmids: a plasmid containing the genes involved in β-carotene synthesis and the genes encoding β-carotene-ketolase and/or a β-carotene-hydrolase and a plasmid containing the gene encoding an apo-carotenoprotein (FIG. 10b).

More preferably, the prokaryote cells are transformed with one plasmid containing the genes involved in β-carotene synthesis, the genes encoding β-carotene-ketolase and/or a β-carotene-hydrolase and the gene encoding an apo-carotenoprotein (FIG. 10c).

As used herein the term “apo-carotenoprotein” or “apo-protein” refers to a protein able to attach a carotenoid. Thus, the apo-carotenoprotein is the protein without the carotenoid.

According to the present invention the apo-carotenoprotein gene may be obtained from any organism selected in the group of any bacteria, any algae, any plant and any animal.

Preferably, the apo-carotenoprotein gene may be obtained from cyanobacteria selected from the group of strains comprising Synechocystis sp., Anabaena sp. or Arthrospira sp.

According to the invention the apo-carotenoprotein is a soluble protein.

Preferably, the apo-carotenoprotein is a soluble protein selected in the group comprising an apo-Orange Carotenoid Protein (OCP), an apo-Red Carotenoid Protein (RCP), an apo-AstaP, an apo-crustacyanin and an apo-glutathione s-transferase like protein (GSTP1).

More preferably, the apo-carotenoproteins of the inventions are selected in the group consisting of apo-OCP and apo-RCP.

According to the more preferred embodiment of the present invention the gene encoding apo-OCP is obtained from bacteria selected in the group consisting in Synechocystis, Arthrospira or Anabaena.

Preferably, the genes sequences of the apo-OCP correspond to the sequences selected from the group comprising SEQ ID NO: 1, SEQ ID NO: 2 and SEQ ID NO: 3.

Preferably, the genes sequences of the apo-RCP correspond to the sequence SEQ ID NO: 4.

According to the present application the sequences of primers used for cloning the genes encoding apo-carotenoprotéine may be synthesized by any conventional method using any conventional skills.

Preferably, the sequences of primers used for cloning the genes encoding apo-carotenoprotéine correspond to SEQ ID NO: 29 to SEQ ID NO: 36 and SEQ ID NO: 73 and 74.

According to a preferred embodiment of the method of the present invention, the gene encoding apo-carotenoprotein may be modified. Usually, the gene encoding apo-carotenoprotein is modified by introducing or deleted 9 to 45 nucleotides, preferably 24 to 30 nucleotides just after the first ATG of 5′ end or just before the stop codon of said gene.

Particularly, these modifications are selected in the group of nucleotide sequences corresponding to SEQ ID NO: 8 to SEQ ID NO: 16 as shown in the sequences listing annexed herewith.

In all apo-carotenoproteins gene sequences a HisTag on N terminal (HisTagNter corresponding to SEQ ID NO: 17) or on C terminal (HisTagCter corresponding to SEQ ID NO: 18) may be introduced.

Preferably these gene sequences of apo-carotenoprotein are modified as shown in table 1.

TABLE 1
Modifications of gene sequences encoding carotenoprotein

| Name of modifying | Constructions of modified |
|---|---|
| sequence | genes encoding apo-carotenoproteins |
| NpCDFduet | ATG + SEQ ID NO: 8 + SEQ ID NO: 1 or 2 |
| | or 3 + HisTagCter |
| NC15 | ATG + SEQ ID NO: 9 + SEQ ID NO: 1 + |
| | HisTagCter |
| NC11 | ATG + SEQ ID NO: 10 + SEQ ID NO: 1 + |
| | HisTagCter |
| NC9 | ATG + SEQ ID NO: 11 + SEQ ID NO: 1 or 4 + |
| | HisTagCter |
| NC7 | ATG + SEQ ID NO: 12 + SEQ ID NO: 1 + |
| | HisTagCter |
| NC6 | ATG + SEQ ID NO: 13 + SEQ ID NO: 4 + |
| | HisTagCter |
| NC4 | ATG = SEQ ID NO: 14 + SEQ ID NO: 1 + |
| | HisTagCter |
| MIX15 | ATG + SEQ ID NO: 15 + SEQ ID NO: 1 + |
| | HisTagCter |
| C9 | ATG + SEQ ID NO: 16 + SEQ ID NO: 1 + |
| | HisTagCter |
| HisTagNter3aa | ATG + SEQ ID NO 17 + SEQ ID NO: 1 or 2 or 3 |
| (amino acid) | |
| HisTagCter | ATG + SEQ ID NO: 1 or 2 or 3 or 4 + |
| | SEQ ID NO: 18 + codon STOP |

According to the present invention the primer's sequences used for the modification of genes encoding apo-carotenoprotein and the ones used for amplification of these genes correspond to SEQ ID NO: 37 to SEQ ID NO: 68.

The modified gene sequences encoding an apo-protein as described above are used in the method of the invention allowing thus increasing the attachment of the carotenoid to apo-protein and also increasing the yield of carotenoid-protein complex.

According to the present invention, the plasmids containing genes involved in carotenoid synthesis and the plasmids containing genes involved in apo-carotenoprotein synthesis contain different replication origins and different selective pressure.

As used in the invention the term “replication origin” refers to a particular sequence in a genome at which replication is initiated bidirectionally or unidirectionally.

As used herein the term “selective pressure” refers to the presence of genes giving resistance to different antibiotics to maintain the plasmids in the prokaryote cells.

According to the preferred embodiment of the present invention when the genes involved in carotenoid synthesis are cloned in the same plasmid or when the genes involved in carotenoid synthesis and the gene encoding an apo-carotenoprotein are cloned in the same plasmid the selective pressure may be replaced by a toxin/anti-toxin system such allowing to avoid the presence of antibiotics in the culture medium.

According to one embodiment of the present Invention genes encoding enzymes involved in carotenoid synthesis and genes encoding an apo-carotenoprotein must be amplified prior to prokaryote cell transfection. Preferably, said genes are amplified by polymerase chain reaction (PCR). The primer's sequences for the amplification correspond to SEQ ID NOs: 29, 30, 31, 32, 33, 34, 35, 36, 39, 40, 57, 58, 61 and 62.

According to the method of the present invention the transformed prokaryote cell are cultured in conditions allowing sequential gene expression.

To obtain a functional carotenoid-protein complex in high quantities, it is very important to cultivate the transfected prokaryote cells such as to induce in the first time the genes expression of genes encoding carotenoids and then, induce the gene expression of a gene encoding an apo-carotenoprotein without stopping or reducing the expression of genes encoding carotenoids.

According to one embodiment of the method of the present invention step b) of culturing the prokaryote cells comprises the following steps:

b1) expressing the genes encoding β-carotene without specific induction;

b2) inducing the gene expression of β-carotene ketolase and/or β-carotene hydrolase at temperature ranges of 33 to 40° C., preferably of 35 to 38 and more preferably about 37° C.;

b3) inducing the gene expression of apo-carotenoprotein at temperature ranges of 20 to 30° C., preferably of 22 to 28° C. and more preferably of 24 to 26° C.

According to a preferred embodiment of the invention, to obtain Synechocystis echinenone attached OCP, the step b2) of inducing β-carotene ketolase genes expression (CrtO) is performed at 37° C. and step b3) of inducing the gene expression of apo-OCP is performed at 28° C.

According to the method of the present invention isolating the carotenoid-protein complex in step c) may be performed by using any conventional column well in the art.

In respect to used tag, the skilled artisan may determinate easily a suitable conventional column to isolate the carotenoid-protein complex.

Preferably, to isolate the carotenoid-protein complex in step c) of the method of the present invention a Nickel column is used when the protein has a Histag.

In one aspect, the present invention relate to a carotenoid-protein complex (or carotenoprotein) obtained by the method of the present invention.

Preferably, said carotenoid-protein complex is a soluble carotenoid-protein complex.

More preferably, said carotenoid-protein complex is a soluble carotenoid-protein complex selected from the group comprising an orange carotenoid protein (OCP), a red carotenoid protein (RCP), AstaP, crustacyanin, glutathione s-transferase like protein (GSTP1).

According to a preferred embodiment of the invention said carotenoid-protein complex is a soluble protein selected from the group comprising an orange carotenoid protein (OCP), a red carotenoid protein (RCP).

According to more preferred embodiment, the carotenoid-protein complex obtained by the method of the present invention is the OCP.

The Orange Carotenoid Protein (OCP) is a soluble protein that attaches a molecule of the ketocarotenoid 3′-hydroxy-echinenone. It is genetically encoded in many species and strains of cyanobacteria (Kirilovsky and Kerfeld, 2012). In cyanobacteria, the OCP is involved in the induction of a photoprotective mechanism that by increasing the thermal dissipation of the excess absorbed energy at the level of the phycobilisome, the cyanobacterial antenna, decreases the energy arriving at the photosynthetic reaction centers (Wilson et al., 2006). The OCP is a photoactive protein. Strong light triggers the activation of the OCP inducing conformational changes in the carotenoid and the protein (Wilson et al., 2008). These changes convert the inactive dark orange OCP in the active red OCP. Only the active red OCP is able to bind the core of phycobilisomes. The bound redOCP quenches the absorbed light energy and the phycobilisome fluorescence with a high efficiency (Kirilovsky and Kerfeld, 2012). The OCP dissipates excess energy into heat.

Recently, it was demonstrated that OCP also protects cyanobacteria cells from oxidative stress by directly quenching singlet oxygen (Sedoud et al., 2014).

In one aspect the present invention also relates to modified apo-carotenoprotein genes encoding modified carotenoid-protein complex. These modified genes are also used in the method of the present invention as genes encoding the apo-protein.

Preferably, the invention relate to a modified gene encoding apo-OCP or apo-RCP characterized in that it is modified by introducing 9 to 45 nucleotides preferably 24 to 30 nucleotides just after the first ATG of 5′ end and/or just before the stop codon of said gene.

According to a preferred embodiment of the invention, the modifications are selected in the group of sequences comprising: SEQ ID NO: 8 to SEQ ID NO: 16 and SEQ ID NO 17 and 18 corresponding to HisTAgNter and HisTAgCter respectively.

Preferably, when the modified gene encodes an apo-OCP (orange carotenoid protein), the modifications are selected in the group of sequences comprising SEQ ID NOs: 8, 9, 10, 11, 12, 14, 15, 16, 17 and 18 (as shown on table 2).

Preferably, when the modified gene encodes an apo-RCP red carotenoid protein), the modifications are selected in the group of sequences comprising SEQ ID NOs: 9, 13 and 18 (as shown on table 2).

In one aspect the present invention relate to a vector, a plasmid or a host cell comprising a modified apo-carotenoprotein according to the invention.

Preferably, said vector according to the invention is a cloning or an expression vector.

The vector can be viral vector such as bacteriophages or non-viral vector such as plasmid.

The host cell according to the invention comprises a nucleic acid molecule according to the invention or vector according to the invention. The host cells according to the invention may be useful for synthesis of polypeptides according to the invention.

In another aspect the invention relate to modified OCP or RCP encoded by the modified apo-protein genes according to the invention.

The method of the present invention has the following advantages:
allows to obtain between 10 to 120 mg carotenoprotein per liter of culture in only 4 days while in the prior art only 1.33 mg per liter after 3 weeks was obtained;
allow to produce numerous natural protein-carotenoid complexes as well as create new ones;
for the first time a water soluble carotenoid protein is obtained in prokaryote cell such as E. coli with attached carotenoid, even with soluble carotenoid proteins wherein the isolated carotenoids are lipid-soluble and present only in membranes.

The present invention will be illustrated by following figures and examples.

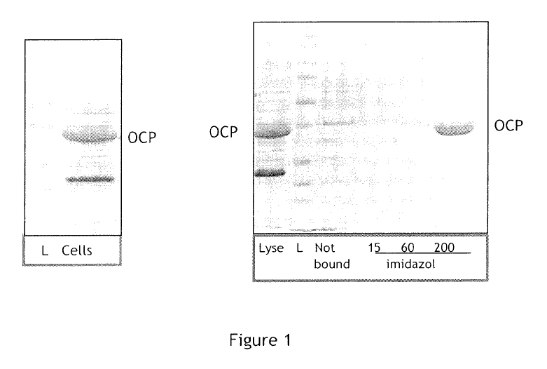

FIG. 1: SDS gels electrophoresis showing the proteins present in E. coli cells overexpressing Arthrospira OCP and the proteins present in the different fractions during the Arthrospira OCP isolation from E. coli cells.

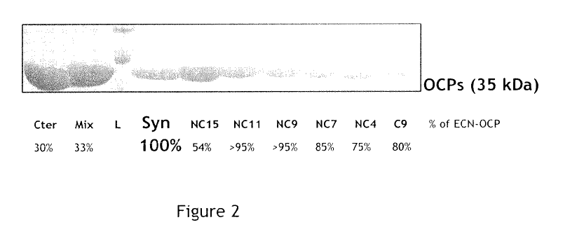

FIG. 2: SDS gels electrophoresis showing the isolated Synechocystis OCPs containing different modifications in N-terminal and/or C-terminal of the protein.

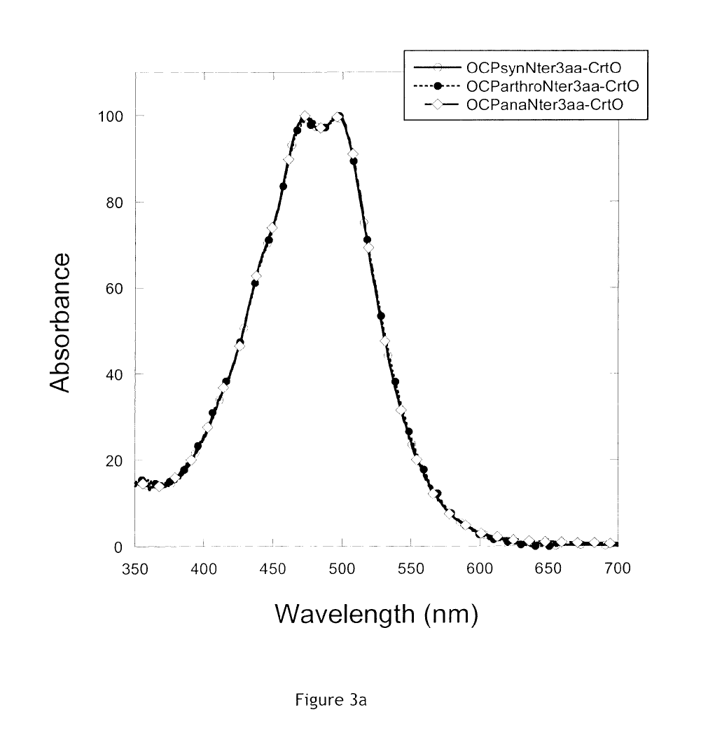

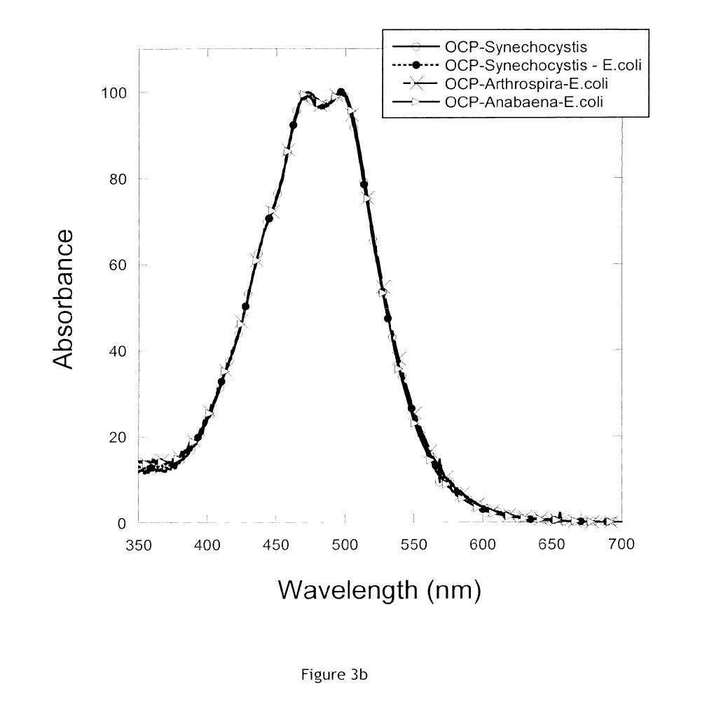

FIG. 3: shows the absorbance spectra of Synechocystis, Arthrospira and Anabaena ECN-OCPs isolated from E. coli cells (FIG. 3a) containing the additional sequence NpCDFduet in the N-terminal. Comparison of the ECN-OCPs isolated from Synechocystis and E. coli cells (FIG. 3b).

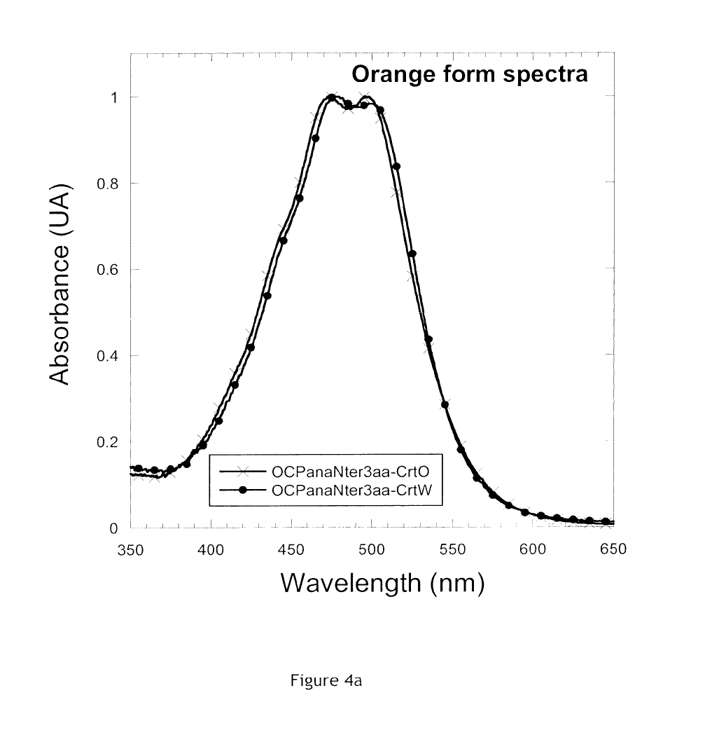

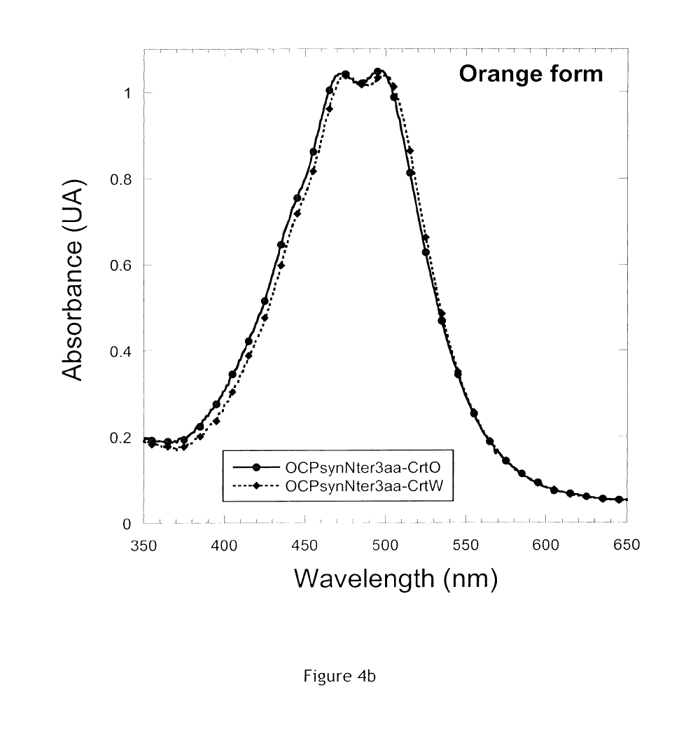

FIG. 4: shows the absorbance spectra of Anabaena (a) and Synechocystis (b) OCPs carrying canthaxanthin and those carrying the echinenone.

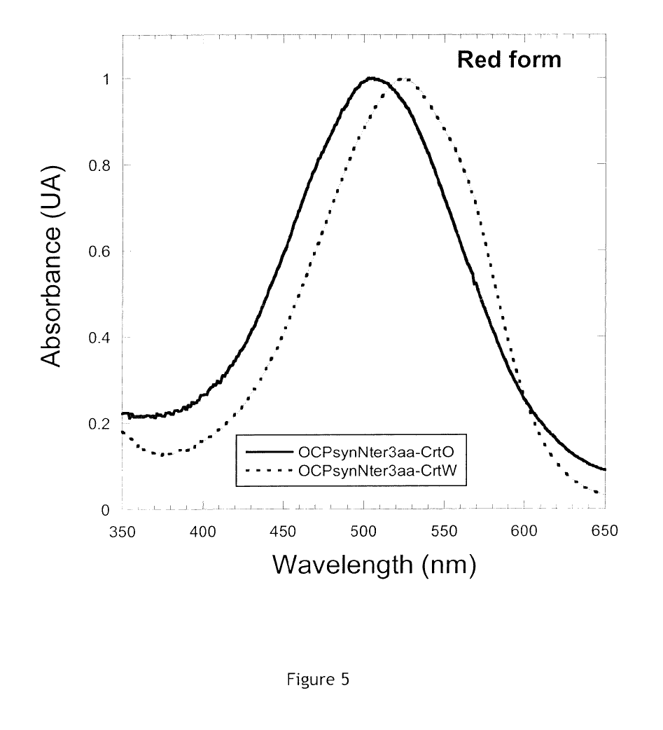

FIG. 5: shows the absorbance spectra of the red Synechocystis OCPs isolated from E. coli cells containing echinenone or canthaxanthin.

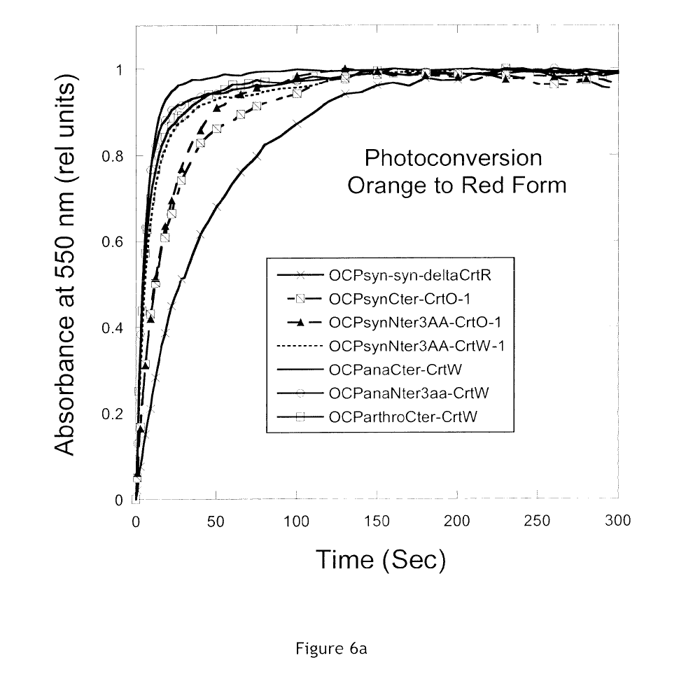

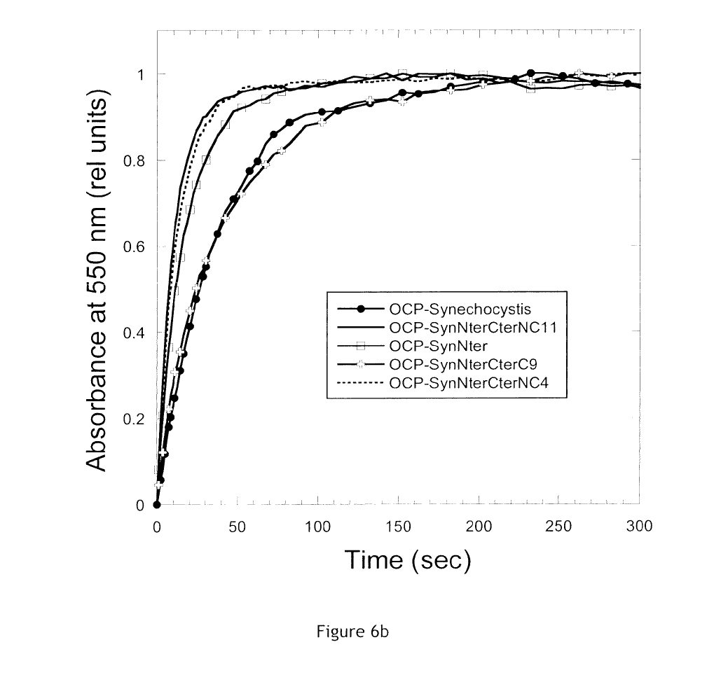

FIG. 6: shows the kinetics of the photoconversion of different modified orange OCP from Arthospira, Anabaena and Synechocystis (FIG. 6a) and different modified orange OCP only from Synechocystis (FIG. 6b) to red OCP.

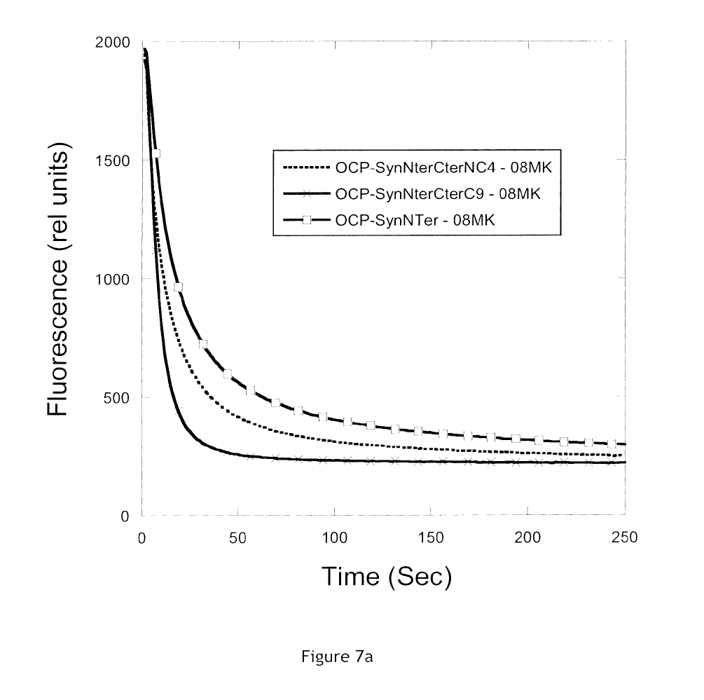

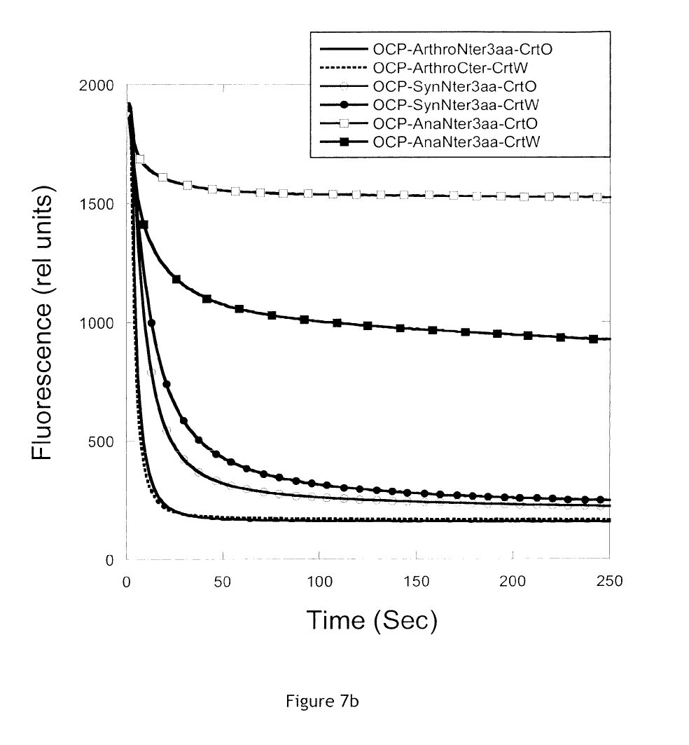

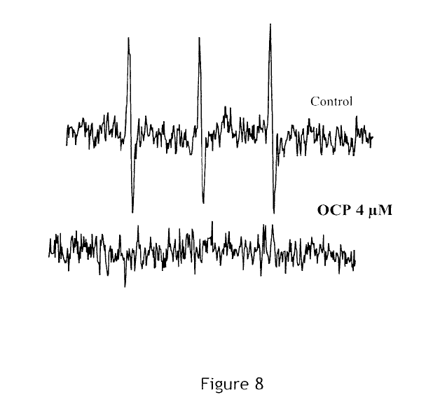

FIG. 7: shows the induction of fluorescence quenching by different OCPs genes from Synechocystis having N-terminal modification (FIG. 7a) and by different OCP genes from Arthospira, Anabaena and Synechocystis having N-terminal modification (FIG. 7b).

FIG. 8: shows EPR (electron paramagnetic resonance) signal of TEMPO (nitroxide radical of TEMPD-HCl 2,2,6,6-tetramethylpiperidone) in the absence and presence of OCP.

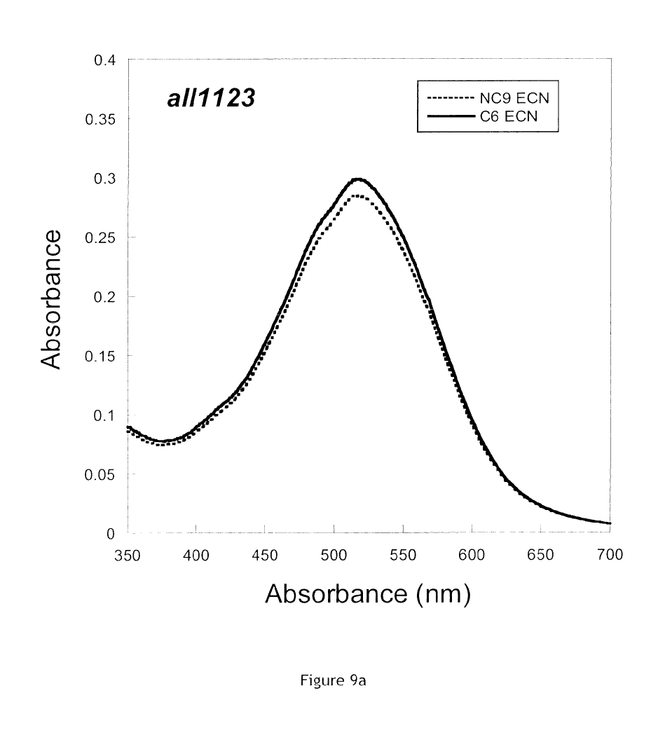

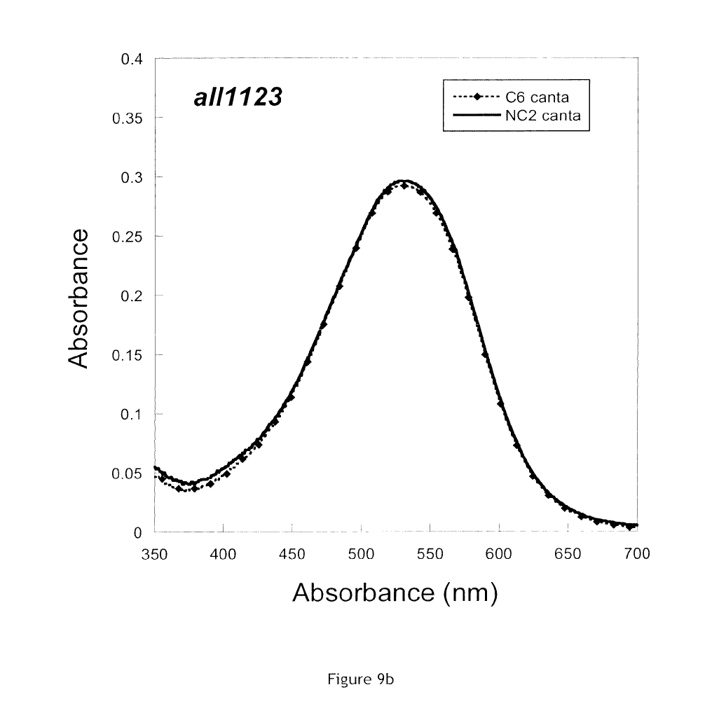

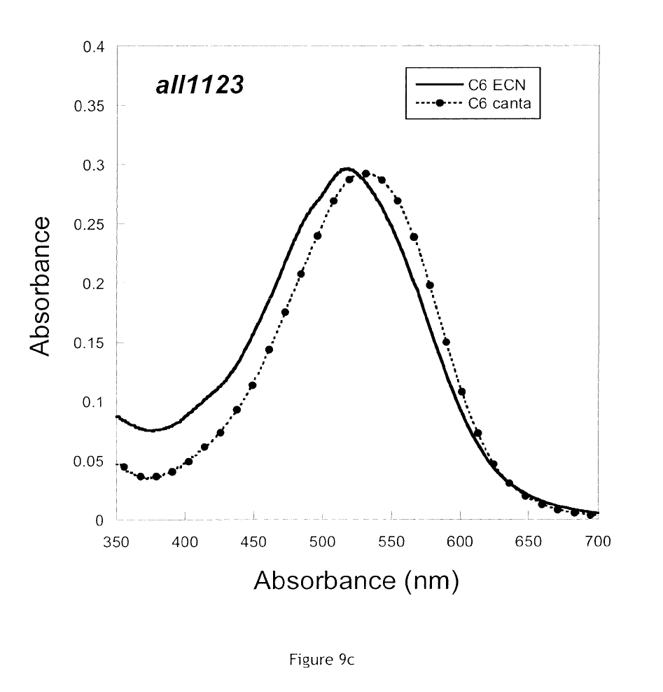

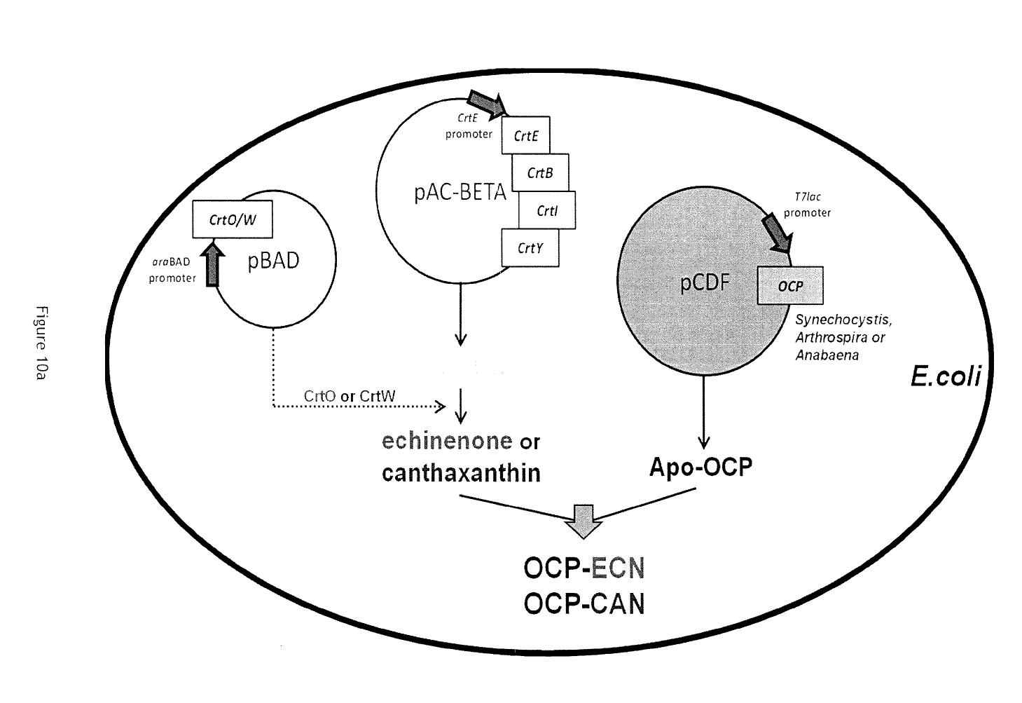

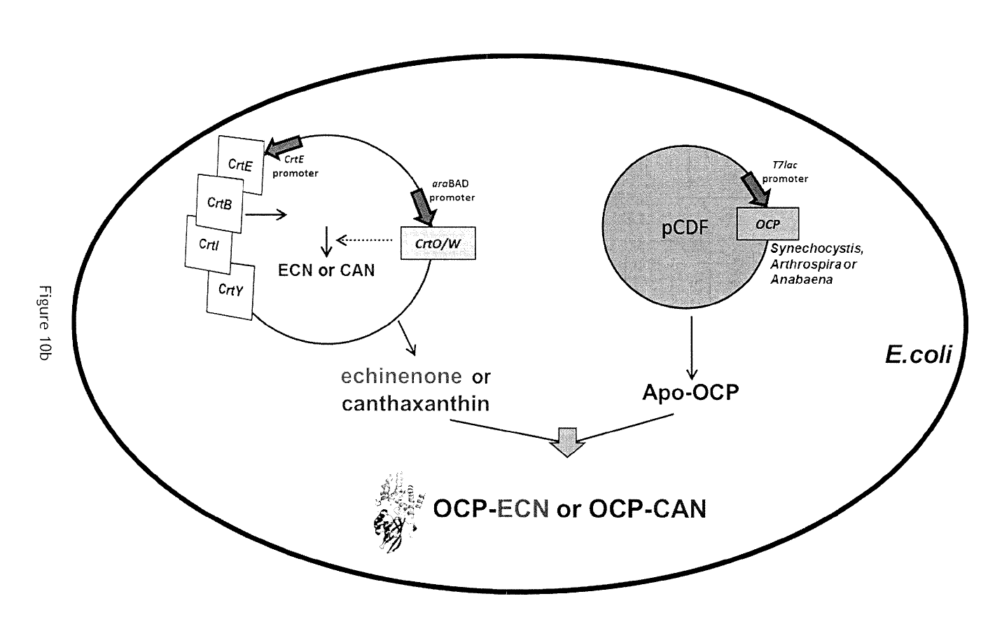

FIG. 9: shows absorbance spectra of RCPs with different modifications in the N-terminal and binding echinenone (FIG. 9a) or cantaxanthin (FIG. 9b) or cantaxanthin and echinenone FIG. 9c).

FIG. 10: shows a schema of the prokaryote cell (E. coli) as performed in the method of the invention, wherein FIG. 10a shows an E. coli cell containing three plasmids, one containing the genes involved in β-carotene synthesis, the second containing the gene encoding β-carotene-ketolase and the third, containing the gene encoding the apo-ocp; FIG. 10b shows an E. coli cell containing two plasmids one containing the genes involved in β-carotene synthesis and containing the gene encoding β-carotene-ketolase and the second, containing the gene encoding the apo-ocp; FIG. 10c shows an E. coli cell containing one plasmid containing the genes involved in β-carotene synthesis, the gene encoding β-carotene-ketolase and the gene encoding the apo-ocp.

### EXAMPLES

### Example 1: Construction of an E. coli Strain Carrying Three Plasmids, One Containing the Genes Involved in β-Carotene Synthesis, the Second Containing the Genes Encoding a β-Carotene-Ketolase and/or a β-Carotene-Hydrolase and the Third Containing a Gene Encoding an Apo-Carotenoprotein

I. Materials and Methods

1) Amplification of Crt Genes Encoding Enzymes Involved in Carotenoid Synthesis and of Carotenoid Binding Proteins (Ocp and Rcp)

Carotenoid Binding Proteins (Apo-Carotenoproteins)
The ocp gene encoding the OCP from three different cyanobacteria strains (slr1963 in Synechocystis PCC 6803 (SEQ ID NO:1); NIES39_N00720 in Arthrospira platensis (SEQ ID NO:2) and all3149 in Anabaena PCC 7120 (SEQ ID NO:3)) and rcp gene (all1123) from Anabaena PCC 7120 (SEQ ID NO:4) were amplified by PCR.
Cyanobacteria genomes and plasmids containing the different ocp genes or the rcp genes with addition of a sequence (CACCACCACCACCACCAC, called HisTagCter) encoding six histidines in the 3′end followed by stop codon were used as templates.
For example such sequences are SEQ ID NO: 1—HisTagCter; SEQ ID NO:2—HisTagCter; SEQ ID NO:3—HisTagCter and SEQ ID NO: 4—HisTagCter.

Crt Enzymes
The crtO gene (slr0088) from Synechocystis PCC 6803 SEQ ID NO:5) and the crtW gene (alr3189) from Anabaena PCC 7120 (SEQ ID NO: 6), both encoding for β-carotene ketolases and the crtR gene (sll1468) of Synechocystis PCC 6803 (SEQ ID NO: 7) encoding for the β-carotene hydrolase were amplified by PCR using cyanobacteria genomes as template and synthetic oligonucleotides as primers (SEQ ID NOs: 19/20 and 21/22 for crtO gene, SEQ ID NOs: 25/26 for crtR gene and SEQ ID NO: 27/28 for crtW gene).
The iProof High Fidelity DNA Polymerase (from Bio Rad) was used to PCR amplification of the desired genes. The primers used herein.

2) Construction of Plasmids: Cloning of Different Ocp, Rcp and Crt Genes

Plasmid Containing the Genes Encoding the Enzymes Needed to β-Carotene Synthesis
Two different plasmids were used:
1) The construction of the Plasmid pAC-BETA, which contains the crtB, crtE, crtl and crtY genes from Erwina herbicola (SEQ ID NO: 75) under the control of the promoter of crtE (gift of Prof Francis X. Cunningham) is described in (Cunningham et al., 1996).
2) The plasmid pACCAR16ΔcrtX containing the crtB, crtE, crtl and crtY genes from Erwina uredovora (SEQ ID NO: 70) under the control of the promoter of crtE, (gift of Prof Sandmann) is described in (Misawa et al., 1995). These plasmids allow the synthesis of β-carotene in the cell.

Plasmids Containing the β-Carotene Ketolases and β-Carotene Hydrolases
The crtO, crtW and crtR genes were cloned in a modified plasmid pBAD/gIII A (from Invitrogen) which contains an arabinose inducible promoter (araBAD) and Ampicillin resistance. The expression of the Crt genes is thus enhanced by arabinose induction in the medium of culture.
CrtO and CrtW catalyse the conversion of β-carotene to echinenone and canthaxanthin respectively and CrtR catalyzes the conversion of β-carotene to zeaxanthin. The plasmid pBAD/gIII A was first modified to avoid the export of the recombinant protein into the periplasmic space of the cells. To this purpose the region encoding the “gene III signal sequence” was deleted. The primers used for the PCR mutagenesis were pBAD/gIIIAmut (F and R) corresponding to SEQ ID NO: 23 and SEQ ID NO: 24.
The plasmid pBAD/gIII A modified have been named pBAD. The Plasmid pBAD was digested with BglII and EcoRI restriction enzymes to clone the crtO gene of Synechocystis or with NcoI and EcoRI restriction enzymes to clone the crtW gene of Anabaena PCC7120 and with NcoI and XhoI restriction enzyme to clone the crtR gene of Synechocystis. Primers CrtO (SEQ ID NOs: 19, 20, 21, 22), primers CrtR (SEQ ID NO: 25 and SEQ ID NO: 26) and primers CrtW (SEQ ID NO: 27 and SEQ ID NO: 28) were used to amplify crtO, crtR and crtW genes respectively. The resulting plasmids were named pBAD-CrtO, pBAD-CrtR and pBAD-CrtW. Theses primers and plasmids are shown on table 2 below.

TABLE 2
Plasmids and primers used for mutagenesis and cloning of
genes encoding β-carotene ketolases and hydrolases

| Plasmids/Restriction | | |
|---|---|---|
| enzyme | Primer's name | Primers SEQ ID NO: |
| pBADgIII-CrtO | CrtO F | 19 |
| BgIII/EcoRI | CrtO R | 20 |
| pBAD-CrtO | | 21 |
| | | 22 |
| pBAD | pBAD/gIIIAmut F | 23 |
| | pBAD/gIIIAmut F | 24 |
| pBAD-CrtR NcoI/XhoI | CrtR F | 25 |
| | CrtR R | 26 |
| pBAD-CrtW | CrtW F | 27 |
| NcoI/EcoRI | CrtW R | 28 |

### Plasmids Containing the Genes of Carotenoid Binding Proteins

The ocp and rcp genes were cloned in a Plasmid pCDFDuet-1 (from Novagen). The plasmid pCDFDuet-1 contains T7lac promoters and Streptomycin/Spectinomycin resistance. The expression of the ocp and rcp genes is thus enhanced by IPTG induction in the medium of culture.
The pCDFduet-1 plasmid was digested with EcoRI and NotI to clone the different ocp genes (from Synechosystis PCC6803, Arthrospira Platensis PCC7345 and Anabaena PCC 7120). The primers OCPsynHistagNter15 (F and R) corresponding to SEQ ID NO: 29 and SEQ ID NO: 30 respectively were used to amplify the Synechocystis ocp gene (1104 nucleotides) using genomic DNA of Synechocystis PCC6803 as template. The primers OCPanaHisTagNter15 (F and R) corresponding to SEQ ID NO: 33 and SEQ ID NO: 34 respectively were used to amplify the Anabaena ocp gene (1076 nucleotides) using genomic DNA of Anabaena PCC 7120 as template. The primers OCParthroHistagNter15 (F and R) corresponding to SEQ ID NO: 31 and SEQ ID NO: 32 respectively were used to amplify the Arthrospira ocp gene (1355 nucleotides) using the plasmid p0F7345 as template (plasmid constructed by (Jallet et al., 2014)). The resulting PCR products were introduced into pCDFDuet-1 to create the pCDF-OCPsynHistagNter15, pCDF-OCPanaHistagNter15, pCDF-OCParthroHistagNter15 and plasmids. In the OCP isolated from E. coli cells carrying these plasmids, an extension of 15 amino acids will be present in the N-terminal of the OCP protein. This extension contains a His-tag of 6 His. (NpCDFduet extension corresponding to SEQ ID NO: 8).

TABLE 3
Plasmids and primers used for cloning of genes encoding ocp genes

| | | Primers |
|---|---|---|
| Plasmids/Restriction | | SEQ ID |
| enzymes | Primer's name | NO: |
| pCDF-OCPsynHisTagNter15 | OCPsynHistagNter15 F | 29 |
| EcoRI/NotI | OCPsynHistagNter15R | 30 |
| pCDF-OCParthroHisTagNter15 | OCParthroHistagNter15 F | 31 |
| EcoRI/NotI | OCParthroHistagNter15 R | 32 |
| pCDF-OCPanaHisTagNter15 | OCPanaHistagNter15 F | 33 |
| EcoRI/NotI | OCPanaHistagNter15 R | 34 |
| pCDFDuet-NC2-1123HIS | RCPanaCter F | 35 |
| NcoI/NotI | RCPanaCter R | 36 |

To obtain a C-terminal His-tagged Synechocystis OCP, first, the GCC sequence coding for Ala73 was mutated to GCG that also code for in alanine to abolish the NcoI site in the Synechocystis ocp gene sequence in the plasmid SK-OCPsyn-P2A-CterHisTagΔFRP (Wilson et al., 2008), to clone the OCPsyn-HisTagCter between NcoI and NotI sites.
Then, pCDFDuet-1 was digested with NcoI and NotI to excise the N-terminal extension containing the His-tag initially present in this plasmid. The ocp genes containing a C-terminal His-Tag from Synechosystis PCC6803, Arthrospira Platensis PCC7345 and Anabaena PCC 7120 were cloned in the plasmid. The primers OCPsynCter (F and R) corresponding to SEQ ID NO: 39 and SEQ ID NO: 40 respectively were used to amplify the ocp gene tagged in C-terminal domain from the plasmid pSK-OCPsyn-P2A-CterHisTagΔFRP-A73A (Wilson et al., 2008). The primers OCParthroCter (F and R) corresponding to SEQ ID NO: 57 and SEQ ID NO: 58 respectively were used to amplify the ocp gene from the plasmid p0F7345His (Jallet et al, 2014) which contains the ocp gene tagged in the C-terminal domain. The primers OCPanaCter (F and R) corresponding to SEQ ID NO: 61 and SEQ ID NO: 62 respectively were used to amplify the ocp gene from genomic DNA of Anabaena PCC 7120, the C-terminal His-tag was then added by PCR mutagenesis. The resulting PCR products were introduced into pCDFDuet-1 to create the pCDF-OCPsynCter, pCDF-OCParthroCter and pCDF-OCPanaCter plasmids. The OCP isolated from E. coli cells containing these plasmids, will contain a His-tag in their C-terminal (HisTagCter corresponding to SEQ ID NO: 18).
To obtain a C-terminal His-tagged RCP, the plasmid pCDFDuet-1 was digested with NcoI and NotI to excise the N-terminal extension containing the His-tag initially present in this plasmid. Then the rcp gene from Anabaena PCC 7120 containing a C-terminal His-Tag was cloned in the plasmid. The primers RCPanaCter (F and R) corresponding to SEQ ID NO: 35 and SEQ ID NO: 36 respectively were used to amplify the rcp gene tagged in the C-terminal from the plasmid pPSBA2-1123HIS (constructed by Rocio Lopez Igual in the lab). The resulting PCR products were introduced into pCDFDuet-1 to create the pCDFDuet-NC2-1123HIS plasmid. The RCP isolated from E. coli cells containing this plasmid, will contain a His-tag in its C-terminal (SEQ ID NO: 18).
Table 4 shows the plasmids used in the present invention, their characteristics and their source.

TABLE 4
All plasmids used in the present invention

| | | Source and |
|---|---|---|
| Plasmid | Characteristics | reference |
| pCDFDuet-1 | Commercially supplied overexpression | Novagen |
| | plasmid vector using T7 promoter capable | |
| | to carry two genes by two multi-cloning | |
| | site, (SmR) | |
| pCDF-OCPsynHisTagNter15 | pCDFDuet-1 derivative, His-tag N-terminal | Performed in |
| | OCP Synechocystis PCC6803 gene | the present |
| | | invention |
| pPSBA2-OCP/FRPsynC- | Plasmid constructed by Adjélé Wilson, | Wilson et al, |
| terHisTAG | which contains the OCP and FRP genes | 2008 |
| | tagged in C-terminal from Synechosystis | |
| | PCC6803 | |
| pCDF-OCPsynCter | pCDFDuet-1 derivative, His-tag C-terminal | Performed in |
| | OCP Synechocystis PCC6803 gene | the present |
| | | invention |
| pOF7345 | Plasmid constructed by Denis Jallet, which | Jallet et al, |
| | contains the OCP and FRP genes of | 2014 |
| | Arthrospira Platensis PCC7345 | |
| pCDF-OCParthroHisTagNter15 | pCDFDuet-1 derivative, His-tag N-terminal | Performed in |
| | OCP Arthrospira Platensis PCC7345 gene | the present |
| | | invention |
| pOF7345His | Plasmid constructed by Denis Jallet, which | Jallet et al, |
| | contains the OCP and FRP genes tagged in | 2014 |
| | C-terminal, of Arthrospira Platensis | |
| | PCC7345 | |
| pCDF-OCParthroCter | pCDFDuet-1 derivative, His-tag C-terminal | Performed in |
| | OCP Arthrospira Platensis PCC7345 gene | the present |
| | | invention |
| pCDF-OCPanaHisTagNter15 | pCDFDuet-1 derivative, His-tag N-terminal | Performed in |
| | OCP Anabaena PCC 7120 gene | the present |
| | | invention |
| pCDF-OCPanaCter | pCDFDuet-1 derivative, His-tag C-terminal | Performed in |
| | OCP Anabaena PCC 7120 gene | the present |
| | | invention |
| pCDFDuet-NC2-1123HIS | pCDFDuet-1 derivative, His-tag C-terminal | Performed in |
| | RCP Anabaena PCC 7120 gene | the present |
| | | invention |
| pBAD/gIII A | Commercially supplied overexpression | Invitrogen |
| | plasmid vector using araBAD promoter, | |
| | (AmpR) | |
| pBAD | pBAD/gIII A derivative, Δ geneIII signal | Performed in |
| | sequence | the present |
| | | invention |
| pBAD-CrtO | pBAD derivative, CrtO Synechocystis | Performed in |
| | PCC6803 gene | the present |
| | | invention |
| pBAD-CrtR | pBAD derivative, CrtR Synechocystis | Performed in |
| | PCC6803 gene | the present |
| | | invention |
| pBAD-CrtW | pBAD derivative, CrtW Anabaena PCC7120 | Performed in |
| | gene | the present |
| | | invention |
| pAC-BETA | P15A ori, Cm(CmR), pACYC184 | Cunningham |
| | derivative, E. herbicola crt genes | et at. (1996) |
| pACCAR16□crtX | P15A ori, Cm(CmR), pACYC184 | Misawa et al, |
| | derivative, E. uredovora crt genes | 1995 |

Modifications of the Sequences of Ocp and Rcp Genes

To increase the yield of carotenoid-OCP complexes in E. coli cells, modifications were introduced in the sequence of the ocp gene. Several modifications were tested in the Synechocystis ocp gene and then the best ones were introduced in the Arthrospira and Anabaena ocp genes. Modifications were also introduced in the Anabaena rcp gene. The sequences added after the first ATG of the gene correspond to SEQ ID NOs: 9, 10, 11, 12, 14, 15 and 16 (respectively called NC15, NC11, NC9, NC7, NC4, Mix15 and C9). They were introduced by directed mutagenesis, using the pCDF-OCPSynCter plasmid as template and primers corresponding to SEQ ID NOs: (41 and 42), (45 and 46), (47 and 48), (49 and 50), (51 and 52), (43 and 44), (53 and 54) respectively The modification HisTagNter3aa was introduced by directed mutagenesis using the pCDF-OCPSynHisTagNter15 plasmid as template and primers corresponding to SEQ ID NOs: 55 and 56 causing the deletion of a part of the N-terminal prolongation. The modification HisTagNter3aa was also introduced to the ocp genes of Arthrospira and Anabaena using the pCDF-OCParthroHisTagNter15 and pCDF-OCPanaHisTagNter15 plasmids as templates and the primers corresponding to SEQ ID NOs: 59 and 60 and SEQ ID NOs: 63 and 64 respectively.

The modifications corresponding to SEQ ID NOs: 11 and 13 (called NC9 and NC6 respectively) were introduced to the rcp gene using the pCDFDuet-NC2-1123HIS plasmid as template and the primers corresponding to SEQ ID NOs: 65 and 66 and SEQ ID NOs: 67 and 68 respectively.

Table 5 shows the modified plasmids comprising a modified ocp genes.

TABLE 5
Modified plasmids comprising modified ocp genes
performed and used in this invention

| | | Source and |
|---|---|---|
| Plasmid modified | Characteristics | reference |
| pCDF-OCPsynCterNterNC15 | Mutagenesis using pCDF-OCPsynCter | Performed in |
| | | the invention |
| pCDF-OCPsynCterNterNC11 | Mutagenesis using pCDF-OCPsynCter | Performed in |
| | | the invention |
| pCDF-OCPsynCterNterNC9 | Mutagenesis using pCDF-OCPsynCter | Performed in |
| | | the invention |
| pCDF-OCPsynCterNterNC7 | Mutagenesis using pCDF-OCPsynCter | Performed in |
| | | the invention |
| pCDF-OCPsynCterNterNC4 | Mutagenesis using pCDF-OCPsynCter | Performed in |
| | | the invention |
| pCDF- | Mutagenesis using pCDF-OCPsynCter | Performed in |
| OCPsynCterNterMIX15 | | the invention |
| pCDF-OCPsynCterNterC9 | Mutagenesis using pCDF-OCPsynCter | Performed in |
| | | the invention |
| pCDF-OCPsynNter3aa | Mutagenesis using pCDF-OCPsynHisTagNter15 | Performed in |
| | | the invention |
| pCDF-OCParthroNter3aa | Mutagenesis using pCDF- | Performed in |
| | OCParthroHisTagNter15 | the invention |
| pCDF-OCPanaNter3aa | Mutagenesis using pCDF-OCPanaHisTagNter15 | Performed in |
| | | the invention |
| pCDF-NC9-1123HIS | Mutagenesis using pCDFDuet-NC2-1123HIS | Performed in |
| | | the invention |
| pCDF-NC6-1123HIS | Mutagenesis using pCDFDuet-NC2-1123HIS | Performed in |
| | | the invention |

3) The E. coli Strains Producing OCP and RCP

Two different bacterial strains were used for gene cloning and gene expression:

1) E. coli XL10-Gold from Agilent (TetrD(mcrA)183 D(mcrCB-hsdSMR-mrr)173 endA1 supE44 thi-1 recA1 gyrA96 relA1 lac Hte [F′ proAB laclqZDM15 Tn10 (Tetr) Amy Camr]) was used for gene cloning and grown in LB medium at 37° C., and

2) E. coli BL21-Gold (DE3) from Agilent (F-ompT hsdS(rB-mB−) dcm+ Tetr gal A(DE3) endA Hte) was used for OCP production.

E. coli strains producing OCP or RCP constructed in this invention are all derivatives of E coli BL21-Gold (DE3).

BL21 cells were transformed with the pAC-BETA, pBAD-CrtO (or pBAD-CrtW or pBAD-CrtR) and pCDF-OCP or pCDF-RCP plasmids. The latter plasmids contain WT or modified sequences of ocp or rcp genes (table 5).

TABLE 6
Strains of E. coli producing different carotenoids created
in this invention and plasmids used to create these strains
used in the invention.

| Strains | Plasmid contents |
|---|---|
| BL21-pβcarotene | pAC-BETA (Francis X. Cunningham) |
| BL21-pβcarotene | pACCAR16ΔCrtX (Sandmann) |
| BL21echi | pAC-BETA |
| | pBAD-CrtO |
| BL21echiBIS | pAC-BETA |
| | pCDF-CrtO |
| BL21zea | pAC-BETA |
| | pBAD-CrtR |
| BL21cantha | pAC-BETA |
| | pBAD-CrtW |

TABLEAU 7
E coli strains containing_ocp and rcp genes (obtained from different
microorganisms cloned in pCDF-Duet plasmids) and containing crtO or
crtW or CrtR genes cloned in pBAD plasmids.

| | PLASMID CONTENT |
|---|---|
| E coli containing SYNECHOCYSTIS | |
| OCP | |
| BL21echi-OCPsynHisTagNter15 | pAC-BETA |
| | pBAD-CrtO |
| | pCDF-OCPsynHisTagNter15 |
| BL21echi-OCPsynCter | pAC-BETA |
| | pBAD-CrtO |
| | pCDF-OCPsynCter |
| BL21echi-OCPsynNter3aa | pAC-BETA |
| | pBAD-CrtO |
| | pCDF-OCPsynNter3aa |
| BL21echi-OCPsynCterNter15 | pAC-BETA |
| | pBAD-CrtO |
| | pCDF-OCPsynCterNter15 |
| BL21echi-OCPsynCterNter11 | pAC-BETA |
| | pBAD-CrtO |
| | pCDF-OCPsynCterNter11 |
| BL21echi-OCPsynCterNter9 | pAC-BETA |
| | pBAD-CrtO |
| | pCDF-OCPsynCterNter9 |
| BL21echi-OCPsynCterNter7 | pAC-BETA |
| | pBAD-CrtO |
| | pCDF-OCPsynCterNter7 |
| BL21echi-OCPsynCterNter4 | pAC-BETA |
| | pBAD-CrtO |
| | pCDF-OCPsynCterNter4 |
| BL21echi-OCPsynCterC9 | pAC-BETA |
| | pBAD-CrtO |
| | pCDF-OCPsynCterNterC9 |
| BL21echi-OCPsynCterMIX15 | pAC-BETA |
| | pBAD-CrtO |
| | pCDF-OCPsynCterNterMIX15 |
| BL21zea-OCPsynHisTagNter15 | pAC-BETA |
| | pBAD-CrtR |
| | pCDF-OCPsynHisTagNter15 |
| BL21zea-OCPsynCter | pAC-BETA |
| | pBAD-CrtR |
| | pCDF-OCPsynCter |
| BL21cantha-OCPsynCter | pAC-BETA |
| | pBAD-CrtW |
| | pCDF-OCPsynCter |
| BL21cantha-OCPsynNter3aa | pAC-BETA |
| | pBAD-CrtW |
| | pCDF-OCPsynNter3aa |
| E coli containing ANABAEANA OCP | |
| BL21echi-OCPanaHisTagNter15 | pAC-BETA |
| | pBAD-CrtO |
| | pCDF-OCPanaHisTagNter15 |
| BL21echi-OCPanaCter | pAC-BETA |
| | pBAD-CrtO |
| | pCDF-OCPanaCter |
| BL21echi-OCPanaNter3aa | pAC-BETA |
| | pBAD-CrtO |
| | pCDF-OCPanaNter3aa |
| BL21cantha-OCPanaCter | pAC-BETA |
| | pBAD-CrtW |
| | pCDF-OCPanaCter |
| BL21cantha-OCPanaNter3aa | pAC-BETA |
| | pBAD-CrtW |
| | pCDF-OCPanaNter3aa |
| E coli containing ARTHROSPIRA OCP | |
| BL21echi-OCParthroHisTagNter15 | pAC-BETA |
| | pBAD-CrtO |
| | pCDF-OCParthroHisTagNter15 |
| BL21echi-OCParthroCter | pAC-BETA |
| | pBAD-CrtO |
| | pCDF-OCParthroCter |
| BL21echi-OCParthroNter3aa | pAC-BETA |
| | pBAD-CrtO |
| | pCDF-OCParthroNter3aa |
| BL21cantha-OCParthroCter | pAC-BETA |
| | pBAD-CrtW |
| | pCDF-OCParthroCter |
| BL21cantha-OCParthroNter3aa | pAC-BETA |
| | pBAD-CrtW |
| | pCDF-OCParthroNter3aa |
| E coli containing ANABAEANA RCP | |
| BL21cantha-RCPanaCter | pAC-BETA |
| | pBAD-CrtW |
| | pCDFDuet-NC2-1123HIS |
| BL21cantha-RCPanaCterNterNC6 | pAC-BETA |
| | pBAD-CrtW |
| | pCDF-NC6-1123HIS |
| BL21cantha-RCPanaCterNterNC6 | pAC-BETA |
| | pBAD-CrtW |
| | pCDF-NC9-1123HIS |

4) Production of Recombinant OCP

Synechocystis echinenone (ECN)-OCP was obtained by following steps:
1) 1 ml of stock glycerol (600 μl E coli cells+400 μl glycerol) is diluted in 200 ml TB (containing 50 μg/ml chloramphenicol, 50 μg/ml ampicillin, 50 μg/ml streptomycin)
2) Incubation at 37° C. for 3-4 hours until arrive to OD600=0.8.
3) When the culture is at OD600=0.8, 0.02% Arabinose is added to induce the transcription of the CrtO gene. The culture in the presence of arabinose is incubated overnight at 37° C.
4) In the next morning; 800 ml of fresh TB are added to the 200 ml E. coli culture.
5) The cells are incubated at 37° C. in the presence of 0.02% Arabinose until OD600=1-1.5.
6) Addition of IPTG to a final concentration of 0.2 mM IPTG to the culture at OD higher than 1.
7) Incubation overnight in the presence of 0.02% arabinose and 0.2 mM IPTG at 28° C. (For rcp this incubation must be done at 18-20° C.)
8) In the next morning the frozen pellet of cells is resuspended in the lyse buffer containing 40 mM Tris pH 8, 10% glycerol and 300 mM NaCl. After resupension, 1 mM [EDTA, PMSF/DMSO, caproïc acid, benzamidine acid] and Dnase were added in the lyses buffer and the cells were broken in dim light using a “French Press” (twice at 750 psi). The broken cells were centrifuged at 20000 rpm for 30 min at 4° C. and the obtained supernatant was then kept for OCP purification on nickel column. The supernatant was loaded on a column of nickel Probond resin (Invitrogen). After column washing (15 and 60 mM Imidazol) the purified OCP was eluted with 200 mM Imidazol.

### Example 2: Construction of an E. coli Strain Carrying Two Plasmids, One Containing the Genes Involved in β-Carotene Synthesis and the Gene Encoding a β-Carotene-Ketolase and the Second, Containing the Genes Encoding an Apo-Carotenoprotein

Materials and Methods

1) Amplification of Crt Genes Encoding Enzymes Involved in Carotenoid Synthesis
The crt operon containing crtB, crtE, crtl and crtY genes from Erwinia uredovora under the control of the promoter of crtE (SEQ ID NO: 70) was amplified by PCR using the pACCAR16ΔCrtX plasmid as template and synthetic oligonucleotides Crt-pBAD (F and R) as primers (SEQ ID NO: 71 and SEQ ID NO: 72).
2) Construction of Plasmid: Cloning of the Crt Operon in pBAD-CrtO Plasmid
The crt operon containing crtB, crtE, crtl and crtY genes, included the crtE promoter and the endogenous terminator of the operon (SEQ ID NO: 70), was amplified. CrtE promoter enhances the synthesis of β-carotene constitutively in E. coli cells.

The construction of the plasmid pBAD-CrtO is described in example 1. In this plasmid the crtO gene is under the control of the araBAD promoter. The expression of the crtO gene is thus enhanced by arabinose induction.
The pBAD-CrtO plasmid was digested with PmeI and XbaI to clone the crt operon (from Erwinia uredovora). The primers Crt-pBAD (F and R) corresponding to SEQ ID NO: 71 and SEQ ID NO: 72 respectively were used to amplify the crt operon (6008 nucleotides) using pACCAR16ΔCrtX plasmid (see example 1) as template. The resulting PCR product was introduced into pBAD-CrtO to create the pBAD-CrtO-Crt plasmid.

3) The E. coli Strains Producing OCP Using Only Two Plasmids
E. coli strains producing OCP or RCP constructed in this invention are all derivatives of E. coli BL21-Gold (DE3).

BL21 cells were transformed with the pBAD-CrtO-Crt plasmid and the pCDF-OCPsynNter3aa plasmid (table 5). The construction of the pCDF-OCPsynNter3aa plasmid is described in example 1.

4) Production of Recombinant OCP

The production of Synechocystis echinenone (ECN)-OCP was obtained by following the steps described in example 1.

### Example 3: Construction of an E. coli Strain Carrying Only One Plasmid Containing the Genes Involved in β-Carotene Synthesis, the Genes Encoding β-Carotene-Ketolase and the Genes Encoding Apo-Carotenoprotein

Methods and Materials

1) Construction of Plasmid: Cloning of the Ocp Gene in pBAD-CrtO-Crt Plasmid

The ocp gene from Synechocystis PCC 6803 under the T7lac promoter control, was cloned into the pBAD-CrtO-Crt plasmid. T7lac promoter thus enhances the expression of the ocp genes by IPTG induction in the medium of E. coli culture.

The construction of the pBAD-CrtO-Crt plasmid is described in example 2. This plasmid contains the crt operon (containing crtB, crtE, crtl and crtY genes) under the control of the crtE promoter and the crtO gene under the control of the araBAD promoter.

The pBAD-CrtO-Crt plasmid was digested with PciI to clone the T7lac-ocp Synechocystis gene sequence (from Synechocystis PCC 6803). The primers T7lacOCP-pBADfull (F and R) corresponding to SEQ ID NO:73 and SEQ ID NO:74 respectively were used to amplify the T7lacOCP gene sequence (1600 nucleotides for T7lacOCPsynNter3aa) using pCDF-OCPsynNter3aa plasmid as template (Table 5).

The resulting PCR product was introduced into pBAD-CrtO-Crt to create the pBAD-CrtO-Crt-OCPsynNter3aa plasmid.

2) The E. coli Strains Producing OCP Using Only One Plasmids
E. coli strains producing OCP or RCP constructed in this invention are all derivatives of E coli BL21-Gold (DE3).
BL21 cells were transformed with the pBAD-CrtO-Crt-OCPsynNter3aa plasmid.

3) Production of Recombinant OCP
Synechocystis echinenone (ECN)-OCP was obtained by following the steps described in example 1.
   The plasmids used in examples 2 and 3 are given in table 8 below:

TABLE 8
Plasmids according to the examples 2 and 3

| Plasmids | Characteristics |
|---|---|
| pBAD-CrtO-Crt | allowing echinenone synthesis |
| pBAD-CrtO-Crt-OCPsynNter3aa | allowing Synechocystis OCP production |
| | with echinenone synthesis |

Alternatively, in examples 2 and 3 the plasmid (pBAD-CrtW-Crt allowing cantaxanthin synthesis, the plasmid pBAD-CrtW-Crt-OCPsynNter3aa (allowing Synechocystis OCP production with canthaxanthin synthesis), the plasmid pBAD-CrtW-Crt-OCPanaNter3aa (allowing Anabaena OCP production with canthaxanthin synthesis)) and the plasmid pBAD-CrtW-Crt-OCParthroNter3aa (allowing arthrospira OCP production with canthaxanthin synthesis) may also be used.

Methods of Characterisation of OCP

Absorbance Measurements

Absorbance spectra and the kinetics of orange to red OCP photoconversion and dark red to orange OCP reconversion were measured in a Specord 5600 (Analyticjena) spectrophotometer. The kinetics were monitored during illumination of the OCP with 5000 μmol photons m⁻² s⁻¹ of white light and after turn-off the light at 18° C.

Quantification of Protein and Carotenoid

The concentration of proteins was measured by the Bradford method and the concentration of carotenoid was deduced from the absorbance spectrum using the absorption coefficient of echinenone A1^{%}=2158. The concentrations of protein and carotenoid are calculated in μM and the ratio will give the % of apo-OCP attached to the carotenoid molecule.

Gel Electrophoresis

The different steps of the OCP and RCP purification were followed by gel electrophoresis using SDS-PAGE on 12% polyacrylamide in a Tris/MES system (Kashino et al., 2001).

Measurement of Carotenoid Content in E. coli Cells and OCP

To determine the carotenoid content of the cells, an aliquot of E. coli cells was harvested by centrifugation at 14000 rpm for 1 min and washed once with water. The cell pellets were resuspended in 500 μl of acetone and incubated at room temperature for 15 min in the dark. The tubes were centrifuged at 14000 rpm for 15 min, and the supernatant containing carotenoids was transferred to a new tube.

To determine the carotenoid content of OCP, OCP was concentrated with centrifugal filter units (Millipore). The carotenoid was extracted by acetone. After drying carotenoid extracted was resupended in 100% di-ether/100 μl ethanol. Carotenoid content was analysed by thin layer chromatography (TLC) with 90% petroleum ether/10% ethanol as mobile phase.

The carotenoid content of OCPs and RCPs was also analysed by High-Performance Liquid Chromatography (HPLC) and Mass spectrometry as described in (Punginelli et al., 2009).

Fluorescence Measurements

Fluorescence quenching and recovery were monitored with a pulse amplitude modulated fluorometer (101/102/103-PAM; Walz, Effelrich, Germany). All measurements were carried out in a stirred cuvette of 1 cm diameter. Typically, the fluorescence quenching was induced by 870 μmol photons m⁻² s⁻¹ of blue-green light (400-550 nm). All the reconstitution experiments were carried out at 23° C. The phycobilisomes are first illuminated and then redOCP (previously illuminated by strong white light) is added and the decreased of fluorescence is monitored.

¹O₂ Detection by EPR Spin Trapping.

The formation of a nitroxide radical, which is a paramagnetic species arising from the interaction of TEMPD with ¹O₂, was measured by EPR in the absence or presence of different amount of purified proteins in buffer Tris-HCl 100 mM pH 8.0, TEMPD-HCl 100 mM and methylene blue (10 μM). The samples were illuminated for 3 min with strong white light (1000 μmol quanta m⁻² s⁻¹).

### Results

1) Characteristics of Isolated OCPs

OCP Isolation

The OCPs were isolated using affinity Nickel-chromatography. The isolation was followed by gel electrophoresis. FIG. 1 shows one example with the purification of ECN-Arthrospira OCP. The soluble proteins present in E coli cells and the proteins present in the different washings (15, 60 and 200 μM imidazole) of the Nickel column are shown. The last lane in each figure shows the purified protein, eluted with 200 μM imidazole (FIG. 1).

The isolated Synechocystis OCPs containing different modifications in the N-terminal and/or C-terminal of the protein are shown on FIG. 2 wherein in the SDS-gel of total proteins of E coli cells a big band at 35 kDa is observed corresponding to the OCP. The OCPs containing small quantities of echinenone are seen as bigger bands. In each lane was loaded the same quantity of carotenoid

Yield of OCP-Carotenoid Complexes

The yield of OCP-carotenoid complex obtained by the method of the present invention is shown on table 9.

TABLE 9
Examples of yield of OCP obtained from E. coli cells when the
overexpression of the ocp gene was done at 28° C.

| | | Average quantity of |
|---|---|---|
| | | total OCP(holo + |
| | | apo-OCP) |
| OCP produced in E. coli | OCP-carotenoid (%) | (mg/L) |
| OCP Synechocystis PCC | | |
| 6803 | | |
| OCPsynNter15-echi | 30-50 | 18-22 |
| OCPsynCter-echi | 20-40 | 4-6 |
| OCPsynNter + 3aa-echi | >95 | 30-35 |
| OCPsynNter + 3aa-cantha | 75-85 | 8-12 |
| OCPsynCterNterMIX15-echi | 30-40 | 20-22 |
| OCPsynCterNterNC15-echi | 45-55 | 18-21 |
| OCPsynCterNterNC11-echi | >95 | 9-11 |
| OCPsynCterNterNC9-echi | >95 | 15-17 |
| OCPsynCterNterNC7-echi | 75-85 | 19-21 |
| OCPsynCterNterNC4-echi | 70-80 | 8-10 |
| OCPsynCterNterC9-echi | 75-85 | 20-22 |
| OCP Arthrospira PCC 7345 | | |
| OCParthroNter15-echi | 30-40 | 18-22 |
| OCParthroCter-echi | 10-25 | 14-16 |
| OCParthroCter- | 50-60 | 30-35 |
| cantha | | |
| OCParthroNter + 3aa- | 30-40 | 24-26 |
| echi | | |
| OCParthroNter + 3aa- | 60-65 | 28-30 |
| cantha | | |
| OCP Anabaena PCC 7120 | | |
| OCPanaNter15-echi | 10-30 | 4-6 |
| OCPanaCter-echi | — | — |
| OCPanaCter-cantha | 50-60 | 60-70 |
| OCPanaNter + 3aa-echi | 50-60 | 50-60 |
| OCPanaNter + 3aa- | 40-50 | 60-70 |
| cantha | | |
| ECN-OCPsynNter3aa | >95 | 60-70 |
| (two plasmids according to | | |
| the example 2) | | |
| ECN-OCPsynNter3aa | >95 | 120-130 |
| (one plasmid according to | | |
| the example 3) | | |

Production of OCP from modified E. coli strains with apo-OCP gene originated from several cyanobacteria has been compared. The results are shown on table 9. All the Synechocystis OCP contained an C-terminal His-tag with the exemption of the OCPsynNter+3aa-echi OCP and OCPsynNter15-echi OCP. The Synechocystis OCPs non-containing N-terminal prolongations attached very badly the carotenoid protein: only 20-35% of the isolated OCP contained a carotenoid molecule.

In addition, the yield of apo-OCP production was lower in the absence of the N-terminal prolongation. When the N-terminal prolongation contains 15 aa, the stability of the carotenoid binding depends on the sequence of these amino acids: The % of ECN-OCP varied from 30 to 55%. N-terminal extensions of 8 to 10 aa just after the first methionine gave the better % of ECN-OCP and the better yield. The amino acid composition of the extension had little effect.

In conclusion, modification of the apo-OCP is necessary to obtain high quantities of carotenoid-protein complexes with antioxidant activity. The protein without carotenoid has not this activity.

The same effect was observed with Arthrospira OCP and with the Anabaena OCP. For which only 10% of the protein binds the echinenone in the absence of the N-terminal prolongation. Large quantities of Arthrospira and Anabaena OCPs binding a carotenoid were obtained in the presence of canthaxanthin. For this the E coli cells were transformed with a pBAD-CrtW plasmid.

Moreover, these results show that the method of the invention using only two plasmids allows obtaining 60 mg of total OCP with plus of 95% holo-OCP from 1 L of E. coli culture. The ECN-OCPsynNter3aa obtained presented the same characteristics than those of the same OCP obtained with three plasmids.

These results also show that the use of only one plasmid in the method of the invention allows obtaining 120 mg of total OCP with plus of 95% holo-OCP from 1 L of E. coli culture. The ECN-OCPsynNter3aa obtained presented the same characteristics than those of the same OCP obtained with three plasmids.

Absorbance Spectra and Photoconversion

FIG. 3 shows the spectra of three OCPs isolated from E. coli cells containing echinenone. The figure also shows the spectrum of the echinenone-OCP isolated from Synechocystis cells. All the spectra were identical. Moreover all the isolated OCPs isolated from E coli cells presented the same spectra independently of the modification they carried.

In addition, all the OCPs isolated from E coli cells carrying echinenone or canthaxanthine are photoactive like the OCP isolated from Synechocystis. In darkness, they are orange and upper illumination they become red (FIG. 4).

FIG. 4 shows that the spectre of the OCPs carrying canthaxanthin is slightly different than those carrying the echinenone. They are slightly red shifted.

The spectra of the red OCPs containing canthaxanthin were red shifted compared to those containing echinenone (FIG. 5).

The photoconversion of orange OCP to red OCP was evaluated by measuring the changes of absorbance at 550 nm (1=100% red form; 0=100% orange form). The increase in absorbance follows the accumulation of the red form.

The kinetics of photoconversion are shown in FIG. 6. Almost all of the OCPs isolated from E coli cells were converted to the red form faster than the OCP isolated from Synechocystis. The OCPs containing canthaxanthin were converted even faster (FIG. 6).

Induction of Phycobilisome Fluorescence Quenching

In cyanobacteria cells, the main activity of the OCP is the induction of excess energy dissipation as heat. The activated red OCP binds the phycobilisomes and thermally dissipates the energy absorbed by them. This is accompanied by a concomitant decrease of fluorescence. The capacity of OCPs to bind to phycobilisomes and to dissipate excess energy can be measured in vitro using a reconstitution system developed by inventor's laboratory (Gwizdala et al., 2011) using isolated Synechocystis phycobilisomes and isolated OCPs.

The phycobilisomes were illuminated 30 sec with high intensities of blue-green light and then photoactivated red OCPs were added and the decrease of fluorescence was followed in a PAM fluorometer during 300 sec. Examples of induction of fluorescence quenching by different red OCPs are shown on FIG. 7.

FIG. 7 shows the decrease of fluorescence induced by binding of red OCPs to the phycobilisomes. It was observed that the different modifications on the N-terminal side of the OCP provoke slight differences in the kinetics of quenching. The modifications can accelerate or decelerate the rates of quenching. Nevertheless all the Synechocystis OCPs with the tested modifications were able to induce a large phycobilisome quenching.

Arthrospira OCPs carrying echinenone or canthaxanthin are able to induce a very fast fluorescence quenching (FIG. 7b). Anabaena OCPs also induced fluorescence quenching. The lower capacity to induce fluorescence quenching of Anabaena OCPs is also observed in an Anabaena OCP carrying hydroxyechinenone and obtained from overexpression in Synechocystis.

Quenching of Singlet Oxygen

The singlet oxygen (¹O₂) quenching activity of OCPs was measured in vitro as described in Sedoud et al, 2014. Electron paramagnetic resonance (EPR) spin trapping was applied for ¹O₂ detection using TEMPD-HCl (2,2,6,6-tetramethyl-4-piperidone). When this nitrone reacts with ¹O₂, it is converted into the stable nitroxide radical, which is paramagnetic and detectable by EPR spectroscopy. The production of ¹O₂ was induced by illumination of the photosensitizer methylene blue. FIG. 8 shows the typical EPR signal of the nitroxide radical obtained after 3 min illumination (1000 μmol quanta m⁻² s⁻¹) of a solution containing methylene blue and TEMPD-HCl. When this incubation was realized in the presence of 4 μM OCP, almost no EPR signal was detected indicating that OCP acts as a ¹O₂ quencher. The concentration of OCP that decreased 50% the EPR signal (I₅₀) was about 1-1.5 μM for all OCPs tested.

2) Characteristics of Isolated RCPs

Yield of RCP-Carotenoid Complexes

In Anabaena PCC 7120 there exist 4 genes coding only rcp genes with a high similarity to the N-terminal domain of the OCP (Sedoud et al., 2014). One of them, all1123, with different modifications in the 5′ end of the gene, was firstly overexpressed in E coli cells in the presence of echinenone or canthaxanthin.

Using the invention described in this document and following the procedure described in example 1 (3 plasmids) four other RCPs were produced in E. coli cells. The all3221, all4783 and all4941 genes from Anabaena PCC7120 (the three other genes of Anabaena) and the tll1269 gene from the cyanobacterium Thermosynechoccocus elongatus encoding for different RCPs were amplified using Anabaena and T elongatus genomic DNA. The amplified genes were cloned in a modified pCDFDuet plasmid to obtain the pcDF-NC2-3221HIS, pcDF-NC2-4783HIS, pcDF-NC2-4941HIS, pcDF-NC2-1269HIS plasmids. BL21 E. coli cells were transformed with each of these plasmids simultaneously with the pAC-BETA and pBAD-CrtW plasmids. The holo-RCPs proteins binding canthaxanthin (A113221-CANTA, A114783-CANTA, A114941-CANTA and T111269) obtained with the procedure described in Example 1 presented the same absorbance spectra than that of the All1123 RCP shown in FIG. 9.

The rcp gene was overexpressed in E coli cells at 28° C. with a sequence coding 6 histidines in the 3′end with or without modifications in the 5′ end. When the gene was induced in E coli cells producing echinenone, less than 5% of the isolated RCP was attached to a carotenoid molecule. Addition of amino acids in its N-terminal did not increase the attachment of the carotenoid. Induction of the rcp gene at lower temperatures did not improve the yield. When the rcp gene was induced in the presence of cantaxanthin, the yield of holo-RCP largely increased arriving to 15-20%. In this case, the induction of the rcp gene at lower temperatures (for example, 20° C.) increased even more the yield of holo-RCP (30-40%).

TABLE 10
shows: % of carotenoid-RCPs obtained from E coli
cells at 28 and 20° C.

| RCP (All1123) produites | RCP- |
|---|---|
| chez E. coli | carotenoid (%) |
| NC2-all1123-ECN 28° C. | 1-3% |
| NC9-all1123-ECN 28° C. | 1-3% |
| C6-all1123-ECN 28° C. | 1-3% |
| C6-all1123-canta 28° C. | 15-20% |
| C6-all1123-canta 20° C. | 30-40% |
| NC2-all1123-canta 20° C. | 30-40% |

The absorbance spectra of the RCP proteins isolated from Synechocystis presented peaks at 498 and 530 nm and a shoulder at 470 nm because they bind mixoxanthophylls while that isolated from Anabaena binding canthaxanthin presented a large pic with a maximum at 530 nm similar to that shown in FIG. 9.

The His-tagged protein of 20 kDa was isolated from E coli cells using a Nickel column as previously described for the isolation of the OCP. The isolated proteins were red. The spectra of the RCPs are described in FIG. 9. The spectra of the cantaxanthin RCPs were red shifted compared to those of the echinenone RCPs.

### COMPARATIVE EXAMPLES

As mentioned above, in order to obtain better attachment of carotenoid to apo-protein in a prokaryote cell and to increase the carotenoid-protein complex yield, a sequential gene expression is essential.

The following examples demonstrate that when the order of gene expression is altered or the synthesis of carotenoids is not maintained when the ocp gene is induced, the carotenoid does not attach the apo-protein and the yield of carotenoid-protein complex decreases.

### Comparative Example 1: Production of Recombinant OCP: (without Arabinose the Second Day)

In this protocol E. coli cells containing the three plasmids described in example 1 were used. The modification of gene encoding OCP corresponds to OCPsynNter3aa-echi which is one of the gene modifications allowing to obtain the best yield of holo-OCP. The only difference between example 1 and comparative example 1 is the steps of gene induction.

To obtain Synechocystis echinenone (ECN)-OCP the following steps were realized:
1) 1 ml of stock glycerol (600 μl E coli cells+400 μl glycerol) is diluted in 200 ml TB (containing 50 μg/ml chloramphenicol, 50 μg/ml ampicillin, 50 μg/ml streptomycin)
2) Incubation at 37° C. for 3-4 hours until arrive to OD600=0.8.
3) When the culture is at OD600=0.8, 0.02% Arabinose is added to induce the transcription of the crtO gene. The culture in the presence of arabinose is incubated overnight at 37° C.
4) In the next morning; 800 ml of fresh TB are added to the 200 ml E. coli culture.
) The cells are incubated at 37° C. until OD600=1-1.5.
6) Addition of IPTG to a final concentration of 0.2 mM IPTG to the culture at OD higher than 1.
7) Incubation overnight in the presence of 0.2 mM IPTG at 28° C. (For rcp this incubation must be done at 18-20° C.)
8) In the next morning the frozen pellet of cells is resuspended in the lyse buffer containing 40 mM Tris pH 8, 10% glycerol and 300 mM NaCl. After resupension, 1 mM [EDTA, PMSF/DMSO, caproïc acid, benzamidine acid] and Dnase were added in the lyses buffer and the cells were broken in dim light using a “French Press” (twice at 750 psi). The broken cells were centrifuged at 20000 rpm for 30 min at 4° C. and the obtained supernatant was then kept for OCP purification on nickel column. The supernatant was loaded on a column of nickel Probond resin (Invitrogen). After column washing (15 and 60 mM Imidazol) the purified OCP was eluted with 200 mM Imidazol.

This protocol gives 15 mg of total OCP and less of 2% holo-OCP (table 11 below). The E. coli cells have a pale colour since the carotenoid is not attached to apo-protein. This colour is completely different to the orange colour of cells containing 100% holo-OCP.

### Comparative Example 2: Production of Recombinant OCP (Simultaneous Induction of Crto and Ocp Genes; Simultaneous Addition of IPTG and Arabinose)

In this protocol E coli cells containing the three plasmids described in example 1 were used. The modification of gene encoding OCP corresponds to OCPsynNter3aa-echi which is one of the gene modifications allowing to obtain the best yield of holo-OCP. The only difference between example 1 and comparative example 1 is the steps of gene induction.

To obtain Synechocystis echinenone (ECN)-OCP the following steps were realized:
1) 1 ml of stock glycerol (600 μl E coli cells+400 μl glycerol) is diluted in 200 ml TB (containing 50 μg/ml chloramphenicol, 50 μg/ml ampicillin, 50 μg/ml streptomycin)
2) Incubation overnight at 37° C.
3) In the next morning; 800 ml of fresh TB are added to the 200 ml E. coli culture.
4) The cells are incubated at 37° C. until OD600=1-1.5.
5) Addition of IPTG to a final concentration of 0.2 mM IPTG and of Arabinose to a final concentration of 0.02% Arabinose to the culture at OD higher than 1.
6) Incubation overnight in the presence of 0.02% arabinose and 0.2 mM IPTG at 28° C. (For rcp this incubation must be done at 18-20° C.)
7) In the next morning the frozen pellet of cells is resuspended in the lyse buffer containing 40 mM Tris pH 8, 10% glycerol and 300 mM NaCl. After resupension, 1 mM [EDTA, PMSF/DMSO, caproïc acid, benzamidine acid] and Dnase were added in the lyses buffer and the cells were broken in dim light using a “French Press” (twice at 750 psi). The broken cells were centrifuged at 20000 rpm for 30 min at 4° C. and the obtained supernatant was then kept for OCP purification on nickel column. The supernatant was loaded on a column of nickel Probond resin (Invitrogen). After column washing (15 and 60 mM Imidazol) the purified OCP was eluted with 200 mM Imidazol.

This protocol gives 18-20 mg of total OCP and less of 2% holo-OCP (table 11 below).

The E. coli cells have a pale colour since the carotenoid is not attached to apo-protein. This colour is completely different to the orange colour of cells containing 100% holo-OCP.

TABLE 11
Yield of carotenoid-protein complex obtained by the method
of comparative examples 1 and 2 compared to the yield of
OCP obtained by the method of the invention.

| | | Average quantity of |
|---|---|---|
| | | total OCP(holo + |
| | | apo-OCP) |
| OCP produced in E. coli | OCP-carotenoid (%) | (mg/L) |
| OCPsynNter + 3aa-echi | <2 | 15 |
| (comparative exemple 1) | | |
| OCPsynNter + 3aa-echi | <2 | 18-20 |
| (comparative exemple 2) | | |
| OCPsynNter + 3aa-echi | >95 | 30-35 |
| (exemple 1) | | |

These results clearly demonstrate that the way of gene induction is essential for obtaining the attachment of the carotenoid to apo-protein and increasing the yield of halo-protein.
Armstrong G (1997) Genetics of Eubacterial Carotenoid Biosynthesis: A Colorful Tale Annu Rev Microbiol 1997, 51: 629-659
Bhosale P, Bernstein P S (2005) Synergistic effects of zeaxanthin and its binding protein in the prevention of lipid membrane oxidation. Biochim Biophys Acta 1740: 116-121
Bourcier de Carbon C, Thurotte A, Wilson A, Perreau F, Kirilovsky D (2015) Biosynthesis of soluble carotenoid holoproteins in E coli cells. Nature Scientific Reports 5, 9085 (doi: 10.1038/srep09085)
Cunningham F X, Jr., Pogson B, Sun Z, McDonald K A, DellaPenna D, Gantt E (1996) Functional analysis of the beta and epsilon lycopene cyclase enzymes of Arabidopsis reveals a mechanism for control of cyclic carotenoid formation. Plant Cell 8: 1613-1626
Giuffra E, Cugini D, Croce R, Bassi R (1996) Reconstitution and pigment-binding properties of recombinant CP29. Eur J Biochem 238: 112-120
Gwizdala M, Wilson A, Kirilovsky D (2011) In vitro reconstitution of the cyanobacterial photoprotective mechanism mediated by the Orange Carotenoid Protein in Synechocystis PCC 6803. Plant Cell 23: 2631-2643
Jallet D, Thurotte A, Leverenz R L, Perreau F, Kerfeld C A, Kirilovsky D (2014) Specificity of the cyanobacterial orange carotenoid protein: influences of orange carotenoid protein and phycobilisome structures. Plant Physiol 164: 790-804
Kashino Y, Koike H, Satoh K (2001) An improved sodium dodecyl sulfate-polyacrylamide gel electrophoresis system for the analysis of membrane protein complexes. Electrophoresis 22: 1004-1007
Kawasaki S, Mizuguchi K, Sato M, Kono T, Shimizu H (2013) A novel astaxanthin-binding photooxidative stress-inducible aqueous carotenoprotein from a eukaryotic microalga isolated from asphalt in midsummer. Plant Cell Physiol 54: 1027-1040
Kerfeld C A, Sawaya M R, Brahmandam V, Cascio D, Ho K K, Trevithick-Sutton C C, Krogmann D W, Yeates T O (2003) The crystal structure of a cyanobacterial water-soluble carotenoid binding protein. Structure 11: 55-65
Kirilovsky D, Kerfeld C A (2012) The orange carotenoid protein in photoprotection of photosystem II in cyanobacteria. Biochim Biophys Acta 1817: 158-166
Misawa, Nakagawa M, Kobayashi K, Yamano S, Izawa Y, Nakamura K and Harashima K, (1990) Elucidation of the Erwinia uredovora carotenoid biosynthetic pathway by functional analysis of gene products expressed in E coli. J Bacteriol, 172, 6704-6712
Misawa N, Satomi Y, Kondo K, Yokoyama A, Kajiwara S, Saito T, Ohtani T, Miki W (1995) Structure and Functional-Analysis of a Marine Bacterial Carotenoid Biosynthesis Gene-Cluster and Astaxanthin Biosynthetic-Pathway Proposed at the Gene Level. Journal of Bacteriology 177: 6575-6584
Punginelli C, Wilson A, Routaboul J M, Kirilovsky D (2009) Influence of zeaxanthin and echinenone binding on the activity of the Orange Carotenoid Protein. Biochim Biophys Acta 1787: 280-288
Sedoud A, Lopez-Igual R, Ur Rehman A, Wilson A, Perreau F, Boulay C, Vass I, Krieger-Liszkay A, Kirilovsky D (2014) The Cyanobacterial Photoactive Orange Carotenoid Protein Is an Excellent Singlet Oxygen Quencher. Plant Cell 26: 1781-1791
Wilson A, Ajlani G, Verbavatz J M, Vass I, Kerfeld C A, Kirilovsky D (2006) A soluble carotenoid protein involved in phycobilisome-related energy dissipation in cyanobacteria. Plant Cell 18: 992-1007
Wilson A, Punginelli C, Gall A, Bonetti C, Alexandre M, Routaboul J M, Kerfeld C A, van Grondelle R, Robert B, Kennis J T, Kirilovsky D (2008) A photoactive carotenoid protein acting as light intensity sensor. Proc. Natl. Acad. Sci. U.S.A 105: 12075-12080
Yasuhiro N, Eiji Y, Wataru (2007), Quenching Activities of Common Hydrophilic and Lipophilic Antioxidants against Singlet Oxygen Using Chemiluminescence Detection System. Carotenoid Science, Vol. 11, 2007, 16-20

Zagalsky P F, Mummery R S, Eliopoulos E E, Findlay J B C (1990) The Quaternary Structure of the Lobster Carapace Carotenoprotein, Crustacyanin—Studies Using Cross-Linking Agents. Comparative Biochemistry and Physiology B-Biochemistry & Molecular Biology 97: 837-848.

1. A method for producing a carotenoid-protein complex in vivo comprising the steps of:
a) transforming of prokaryote cells with genes involved in carotenoid synthesis and a gene encoding an apo-carotenoprotein;
b) culturing of transformed prokaryote cells in step a) in conditions allowing sequential gene expression, wherein the expression of the genes involved in carotenoid synthesis is induced prior to the expression of the gene encoding an apo-carotenoprotein
c) isolating and purifying the carotenoid-protein complex expressed by the prokaryote cells.

2. The method according to claim 1 wherein the prokaryote cell in step a) is transformed with at least one plasmid containing the genes involved in carotenoid synthesis and with one plasmid containing a gene encoding an apo-carotenoprotein, wherein said plasmids contain different replication origins and different selective pressure.

3. The method according to claim 1 wherein the at least one plasmid containing the genes involved in carotenoid synthesis in step a) is a plasmid containing the genes involved in β-carotene synthesis

4. The method according claim 1 wherein the transforming of step a) further comprises a second plasmid containing the genes encoding a β-carotene ketolase and/or the genes encoding a β-carotene hydrolase.

5. The method according claim 1, where in the plasmid containing the genes involved in β-carotene synthesis further contains the genes encoding a β-carotene ketolase and/or the genes encoding β-carotene hydrolase under the control of an inducible promoter, preferably ara promoter.

6. The method of claim 1, wherein in step a) the prokaryotic cell is transformed with one plasmid containing the genes involved in β-carotene synthesis, the genes encoding a β-carotene ketolase and/or the genes encoding β-carotene hydrolase and the gene encoding an apo-carotenoprotein.

7. The method according to claim 1, wherein step b) of culturing the prokaryote cells comprises the following steps:
b1) expressing the genes encoding β-carotene without specific induction;
b2) inducing the gene expression of β-carotene ketolase and/or β-carotene hydrolase at temperature ranges of 33 to 40° C., preferably of 35 to 38 and more preferably about 37° C.;
b3) inducing the gene expression of apo-carotenoprotein at temperature ranges of 20 to 30° C., preferably of 22 to 28° C. and more preferably of 24 to 26° C.

8. The method of claim 1 wherein the apo-carotenoprotein gene is modified by introducing or deleting 9 to 45 nucleotides preferably 24 to 30 nucleotides after the first ATG of 5′ end or before the stop codon of said gene.

9. The method according to claim 8, wherein the modifications after the first ATG of 5′ end are selected from the group of sequences comprising: SEQ ID NOs: 8, 9, 10, 11, 12, 14, 15, 16, 17 and the modification before the stop codon corresponds to SEQ ID NO: 18.

10. The method according to claim 8, wherein the modifications after the first ATG of 5′ end are selected in the group of sequences comprising: SEQ ID NOs: 11, 13, and the modification before the stop codon correspond to SEQ ID NO: 18.

11. The method according to claim 1, wherein the carotenoid genes are obtained from any bacteria, any algae or any plant containing carotenoids, preferably from cyanobacteria and non photosynthetic eubacteria and the said apo-carotenoprotein gene is isolated from any organism, preferably from cyanobacteria.

12. The method according to claim 1, wherein the prokaryote cells are selected in the group of non-photosynthetic prokaryotic cells preferably comprising E. coli or Lactococcus lactis.

13. A carotenoid-protein complex obtained by the method according to claim 1.

14. The carotenoid-protein complex according to claim 13 which is a protein selected in the group of soluble proteins, preferably comprising an orange carotenoid protein (OCP), a red carotenoid protein (RCP), AstaP, crustacyanin, glutathione s-transferase like protein (GSTP1).

15. A modified gene as used in the method according to claim 1 encoding apo-OCP or apo-RCP characterized in that it is modified by introducing 9 to 45 nucleotides, preferably 24 to 30 nucleotides after the first ATG of 5′ end and/or before the stop codon of said gene.

16. A modified gene encoding apo-OCP according to claim 15 wherein the modifications after the first ATG of 5′ end are selected from the group of sequences comprising: SEQ ID NOs: 8, 9, 10, 11, 12, 14, 15, 16, 17 and the modification before the stop codon corresponds to SEQ ID NO: 18.

17. A modified gene encoding apo-RCP according to claim 15, wherein the modifications after the first ATG of 5′ end are selected in the group of sequences comprising: SEQ ID NOs: 11, 13, and the modification just the stop codon corresponds to SEQ ID NO: 18.

18. A vector, a plasmid or a host cell comprising a modified apo-carotenoprotein gene according to claim 15.

19. The modified OCP or RCP encoded by the modified apo-protein gene according to claim 15.


SEQUENCE LISTING (75 sequences)

SEQ ID NO: 1 - DNA, 954 bp, Synechocystis PCC 6803
  misc_feature: OCP
atgccattcaccattgactctgcccgcggaattttccctaacaccctagcggccgacgttgtacccgctaccatcgcccgttttagccaactcaatgccgaagatcaattagctctgatttggtttgcttacctagaaatgggtaaaacccttacgatcgccgcccctggggccgccagcatgcaactggccgaaaatgccctcaaggaaattcaagccatggggcccctccaacaaacccaggccatgtgtgacttggccaaccgagctgacactcccctgtgccgcacctacgccagttggtcccccaacattaaacttggcttctggtaccgtttaggcgaactgatggagcagggctttgtcgcccccattcccgctggttaccaactttctgccaatgccaatgcagtgctagctacgatccaaggcctagaatccggtcaacaaattaccgtattgcgtaatgccgtggtggacatgggcttcaccgctggtaaggacggcaaacgcatcgctgagcccgtagttcctccccaagataccgccagccgcaccaaagtttccattgaaggggtgaccaatgccactgtcctcaactacatggacaacctcaatgccaatgacttcgacaccttaatcgaattgttcacctccgacggtgccctccaaccgcccttccaacggcccattgtcggtaaagaaaatgtgctccgctttttccgggaagagtgccaaaacctgaaattgatcccggaacggggggttactgaacccgctgaagatggtttcacccaaattaaagttaccggtaaggtgcaaaccccttggtttggtggcaacgtgggcatgaatatcgcctggcgctttctcctcaaccccgagggcaaaattttctttgtggcgatcgacctccttgcttcccccaaagaattactcaactttgctcgctag

SEQ ID NO: 2 - DNA, 960 bp, Arthrospira PCC 7145
  misc_feature: OCP
atgccattcaccattgactcggctcgtagcatttttccagaaaccctagcggctgacgtagttccggcgacgatcgctcggttcaagcagcttagtgccgaagatcaactggctctcatttggtttgcgtatctggaaatgggtaaaaccatcaccattgctgctcctggggctgcgaatatgcagtttgctgaaaacaccctacaggaaattcggcagatgacccccctacagcaaacccaggctatgtgtgacctggcgaaccggacagatactcccatttgtcgtacctatgctagttggtctcctaatatcaagctgggtttctggtatgaactagggcgtttcatggatcaaggacttgttgcgcccattcccgaaggctacaaactctcagctaacgccaacgctatcctagtgactattcaaggtattgatccaggtcaacaaatcacggtgctgcgtaattgtgttgttgatatggggttcgataccagcaaattaggcagctatcaacgggtagcagaacctgttgtacctcctcaagagatgtcccaacggactaaagttcaaatcgaaggtgtgaccaattccacagtcctgcaatacatggacaacttgaatgctaacgattttgataacttaattagtctgtttgcagaagatggagctttacaacctcctttccaaaaaccaattgttggtaaagagaatgtgctgcggttcttccgggaagaatgccagaacctgaagttaatcccagaacgaggtgtttctgaaccgacggaagatggctatactcagattaaggttactggtaaagtgcaaactccctggtttggtggtaatgtggggatgaatattgcttggcgcttcttgctgaacccagaaaataaggttttctttgtggcgatcgatttgttagcatctcccaaagagttgttgaacttggtgcgctag

SEQ ID NO: 3 - DNA, 1075 bp, Anabaena PCC 7120
  misc_feature: OCP
atggcaattactatcgattccgcccgtcgtatcttccctaacacattacaggctgatgcagtgccagcgctgactgcacgattcaaccaactcagtgcagaagatcaactggcatggacatggttcgctttcttagagatgggcaaaactattactgttgcagcgcctggtgcagctagtatgcagtttgcggaaggtatcctcaagcaaatcaaggaaatgacctttgaggaacaaactcaggttatgtgcgacctggcaaaccacacagatacgcccatttgccgcacctatgctacttggtcgcctaatatcaagttaggtttttggaatcaactgggagaatggatggagcaaggtgccgttgcgccaattcctgctggctatcagctttcagccaatgctaatgcagtattggaaaccctgaaaagtctggatcaaggacaacagattaccgtcctccgcagttccgttgtagatatgggatttgatgcagctaaactagacggctacacaagagttgctgaaccattagttgcacctaaagatatttcccagcgtgttcaagtcaccattgagggaattaataattccactgtgttgaattacatgaacaacttgaacgccaatgactttgatgaactgatcaagttgttcgtggaggatggagctttgcagcctcccttccaaagaccaatcgttggtaaggatgcaattctgcggttcttccgtgaagaatgccagaacctcaacttgcttccagagcgcggtgtggctgaacccgcagacgatggctatactcaggtgaaagtgacaggtaaagttcagaccccttggtttggtgcagcagtagggatgaacatggcttggcgcttcttgctcaacccccaaggtaaaatcttcttcgtagctattgacttgttagcctctcccaaagaacttttgaacttggttcgctaaaggaaaataacaaaacaactaaatagctgattatcgcggtgagcgctctcttaacgaagggcgctcaattattctgagcaaacgcaattaattaatacaagtttaagattgagtc

SEQ ID NO: 4 - DNA, 492 bp, Anabaena PCC 7120
  misc_feature: RCP
atgacttttacacaaacaaacgatcccactattcgtaaatatgtccaatcatggcagaaattggatgtagacgaacaattggctttgttttggtttatctacaaagaaatgggtggtgcaattacaccagcagcccctggtgctagcactgtttccccagcaattgccgaaggtttatttaatcaagtcaaagaattgaatcatgaacagcaattgcaactacaacgcgacttaattcggcgtgtagataaccaactcagccgcgaatacggctctctgggtgataccactaagctactgttttggtatcttttatctcaaggaatggataatgcaaccatagttccttttcctgccgattataaactgtcttcagaatcccaagaactgttggaaggaattaaaggcttaggttttgaacaacagattactcttttccgtgattacgtttccccaatgggtgctgaagcaaaagcgggtgcggaaatttag

SEQ ID NO: 5 - DNA, 1629 bp, Synechocystis PCC 6803
  misc_feature: CrtO(beta-carotene ketolase)
atgatcaccaccgatgttgtcattattggggcggggcacaatggcttagtctgtgcagcctatttgctccaacggggcttgggggtgacgttactagaaaagcgggaagtaccagggggggcggccaccacagaagctctcatgccggagctatccccccagtttcgctttaaccgctgtgccattgaccacgaatttatctttctggggccggtgttgcaggagctaaatttagcccagtatggtttggaatatttattttgtgaccccagtgttttttgtccggggctggatggccaagcttttatgagctaccgttccctagaaaaaacctgtgcccacattgccacctatagcccccgagatgcggaaaaatatcggcaatttgtcaattattggacggatttgctcaacgctgtccagcctgcttttaatgctccgccccaggctttactagatttagccctgaactatggttgggaaaacttaaaatccgtgctggcgatcgccgggtcgaaaaccaaggcgttggattttatccgcactatgatcggctccccggaagatgtgctcaatgaatggttcgacagcgaacgggttaaagctcctttagctagactatgttcggaaattggcgctcccccatcccaaaagggtagtagctccggcatgatgatggtggccatgcggcatttggagggaattgccagaccaaaaggaggcactggagccctcacagaagccttggtgaagttagtgcaagcccaagggggaaaaatcctcactgaccaaaccgtcaaacgggtattggtggaaaacaaccaggcgatcggggtggaggtagctaacggagaacagtaccgggccaaaaaaggcgtgatttctaacatcgatgcccgccgtttatttttgcaattggtggaaccgggggccctagccaaggtgaatcaaaacctaggggaacgactggaacggcgcactgtgaacaataacgaagccattttaaaaatcgattgtgccctctccggtttaccccacttcactgccatggccgggccggaggatctaacgggaactattttgattgccgactcggtacgccatgtcgaggaagcccacgccctcattgccttggggcaaattcccgatgctaatccgtctttatatttggatattcccactgtattggaccccaccatggccccccctgggcagcacaccctctggatcgaattttttgccccctaccgcatcgccgggttggaagggacagggttaatgggcacaggttggaccgatgagttaaaggaaaaagtggcggatcgggtgattgataaattaacggactatgcccctaacctaaaatctctgatcattggtcgccgagtggaaagtcccgccgaactggcccaacggctgggaagttacaacggcaatgtctatcatctggatatgagtttggaccaaatgatgttcctccggcctctaccggaaattgccaactaccaaacccccatcaaaaatctttacttaacaggggcgggtacccatcccggtggctccatatcaggtatgcccggtagaaattgcgctcgggtctttttaaaacaacaacgtcgtttttggtaa

SEQ ID NO: 6 - DNA, 939 bp, Synechocystis PCC 6803
  misc_feature: CrtR(beta-carotene ketolase)
ctagggcttgtcagatgattgagaattattatggccgtgaaaacgaataccaaggaaaacatcatagaggaagctccagaaatttttcccttccagtaatcccagggattgatcacaacccttctcatccaaaatgggcttggtgatgtgataggtgttttggtactgataccaaggaatagaaggccaaaggtggtggatcaggtggtaattttgcccgaaaatgagccaatttaaaatggggctgggataaaccctggcatttttccaacggttgcgttcttggaaaggtcgatggggcaggtaatcaaaaaacagtcccagggcaatgccaacaattaaagcaggcacaaaccagtaattcatcacaaagccgataaagccgtaatgaatgccgagaaaaacgatcgtgaacaacacaagccgacttaagaaccactctagtagctcatatttgcgccataaccgccgtttaaagaaaaagatctcatggtagaagaaccgggcggcaatgaggaagaggggaccgccggtggaaacaaaatggtctgggtcattttcagggtcattgacgttggcgtggtgttggagatgaacccgggtaaagacgggaaaagcaaagcccaacattaaggcactaccgtggccaagcactgcattaataatggtgttccgatgggccgcattgtgggatgcatcatggatcactgtgcctgacaggtgcagcgccagcaccgaacaactaaaacaaagccagtggggcagcccccattgccagtagccaaaaacgttagcgagcattaagagaatagctatcccgaacatggccacgttgggattgaagccgccgtccgcctgtaaaaactcttttgggacagcttgggaaacggtttgcagcggttgggtcgcctgcattactatgacggactcctggcacac

SEQ ID NO: 7 - DNA, 777 bp, Anabaena PCC 7120
  misc_feature: CrtW(beta-carotene ketolase)
atggttcagtgtcaaccatcatctctgcattcagaaaaactggtgttattgtcatcgacaatcagagatgataaaaatattaataagggtatatttattgcctgctttatcttatttttatgggcaattagtttaatcttattactctcaatagatacatccataattcataagagcttattaggtatagccatgctttggcagaccttcttatatacaggtttatttattactgctcatgatgccatgcacggcgtagtttatcccaaaaatcccagaataaataattttataggtaagctcactctaatcttgtatggactactcccttataaagatttattgaaaaaacattggttacaccacggacatcctggtactgatttagaccctgattattacaatggtcatccccaaaacttctttctttggtatctacattttatgaagtcttattggcgatggacgcaaattttcggattagtgatgatttttcatggacttaaaaatctggtgcatataccagaaaataatttaattatattttggatgataccttctattttaagttcagtacaactattttattttggtacatttttgcctcataaaaagctagaaggtggttatactaacccccattgtgcgcgcagtatcccattacctcttttttggtcttttgttacttgttatcacttcggctaccacaaggaacatcacgaataccctcaacttccttggtggaaattacctgaagctcacaaaatatctttataa

SEQ ID NO: 8 - DNA, 42 bp, Artificial
  NpCDFduet
atgggcagcagccatcaccatcatcaccacagccaggatccg

SEQ ID NO: 9 - DNA, 48 bp, Artificial
  NC15
atgggcagcagcaatcaggctaatcaggtgactctaaatccacaagtt

SEQ ID NO: 10 - DNA, 33 bp, Artificial
  NC11
atgggcagcagcaatcaggctaatcaggtgact

SEQ ID NO: 11 - DNA, 27 bp, Artificial
  NC9
atgggcagcagcaatcaggctaatcag

SEQ ID NO: 12 - DNA, 21 bp, Artificial
  NC7
atgggcagcagcaatcaggct

SEQ ID NO: 13 - DNA, 18 bp, Artificial
  NC6
atgggcagcagcaagagg

SEQ ID NO: 14 - DNA, 12 bp, Artificial
  NC4
atgggcagcagc

SEQ ID NO: 15 - DNA, 48 bp, Artificial
  MIX15
atgggcagcagccgactagacaatccagaaaaaactgacattgaacca

SEQ ID NO: 16 - DNA, 27 bp, Artificial
  C9
atgggcagcagcaaaaagagaagggct

SEQ ID NO: 17 - DNA, 30 bp, Artificial
  HisTagNter
atgggcagcagccatcaccatcatcaccac

SEQ ID NO: 18 - DNA, 18 bp, Artificial
  HisTagCter
caccaccaccaccaccac

SEQ ID NO: 19 - DNA, 30 bp, Artificial
  Primer CrtO F pBADgIII-CrtO
aacagaagatctatcaccaccgatgttgtc

SEQ ID NO: 20 - DNA, 30 bp, Artificial
  Primer CrtO R pBADgIII-CrtO
tctgttgaattcgcttcacttgccatacac

SEQ ID NO: 21 - DNA, 48 bp, Artificial
  Primer pBAD-CrtO
caggaggaattaaccatgatcaccaccgatgttgtcattattggggcg

SEQ ID NO: 22 - DNA, 48 bp, Artificial
  Primer pBAD-CrtO
catggttaattcctcctgttagcccaaaaaacgggtatggagaaacag

SEQ ID NO: 23 - DNA, 48 bp, Artificial
  Primer pBAD/gIIIAmut F pBAD
caggaggaattaaccatgaccatggagctcgagatctgcagctggtac

SEQ ID NO: 24 - DNA, 48 bp, Artificial; identical to SEQ ID NO: 22
  Primer pBAD/gIIIAmut R pBAD

SEQ ID NO: 25 - DNA, 32 bp, Artificial
  Primer CrtR F pBAD-crtR
aacagaccatgggctgccaggagtccgtcata

SEQ ID NO: 26 - DNA, 30 bp, Artificial
  Primer CrtR R pBAD-crtR
tctgttctcgagcaattacttacccgattt

SEQ ID NO: 27 - DNA, 31 bp, Artificial
  Primer CrtW F pBAD-crtW
gatataccatggttcagtgtcaaccatcatc

SEQ ID NO: 28 - DNA, 28 bp, Artificial
  Primer CrtW R pBAD-crtW
gcatcggaattcagtcgtattccagcag

SEQ ID NO: 29 - DNA, 31 bp, Artificial
  Primer OCPsynHistagNter15 F pCDF-OCPsynHisTagNter15
aacagagaattctccattcaccattgactct

SEQ ID NO: 30 - DNA, 32 bp, Artificial
  Primer OCPsynHistagNter15 R pCDF-OCPsynHisTagNter15
tctgttgcggccgctgtaacattaacttatga

SEQ ID NO: 31 - DNA, 33 bp, Artificial
  Primer OCParthroHistagNter15 F pCDF-OCParthroHisTagNter15
aacagagaattctccattcaccattgactcggc

SEQ ID NO: 32 - DNA, 37 bp, Artificial
  Primer OCParthroHistagNter15 R pCDF-OCParthroHisTagNter15
tctgttgcggccgcttatcattacctaattgagcatt

SEQ ID NO: 33 - DNA, 33 bp, Artificial
  Primer OCPanaHistagNter15 F pCDF-OCPanaHisTagNter15
aacagagaattctgcaattactatcgattccgc

SEQ ID NO: 34 - DNA, 34 bp, Artificial
  Primer OCPanaHistagNter15 R pCDF-OCPanaHisTagNter15
tctgttgcggccgcgactcaatcttaaacttgta

SEQ ID NO: 35 - DNA, 33 bp, Artificial
  Primer RCPanaCter F pCDFDuet-NC2-1123HIS
gatataccatgggcacttttacacaaacaaacg

SEQ ID NO: 36 - DNA, 35 bp, Artificial
  Primer RCPanaCter R pCDFDuet-NC2-1123HIS
cattatgcggccgcctaatgatgatgatgatgatg

SEQ ID NO: 37 - DNA, 28 bp, Artificial
  Primer pCDF-OCPsynCter NcoI/NotI
caaggaaattcaagcgatggggcccctc

SEQ ID NO: 38 - DNA, 28 bp, Artificial
  Primer pCDF-OCPsynCter NcoI/NotI
gaggggccccatcgcttgaatttccttg

SEQ ID NO: 39 - DNA, 29 bp, Artificial
  Primer OCPsynCter F
aacagaccatggcattcaccattgactct

SEQ ID NO: 40 - DNA, 32 bp, Artificial; identical to SEQ ID NO: 30
  Primer OCPsynCter R

SEQ ID NO: 41 - DNA, 57 bp, Artificial
  Primer pCDF-OCPsynCterNterNC15
aatcaggctaatcaggtgactctaaatccacaagttccattcaccattgactctgcc

SEQ ID NO: 42 - DNA, 60 bp, Artificial
  Primer pCDF-OCPsynCterNterNC15
aacttgtggatttagagtcacctgattagcctgattgctgctgcccatggtatatctcct

SEQ ID NO: 43 - DNA, 57 bp, Artificial
  Primer pCDF-OCPsynCterNterMIX15
cgactagacaatccagaaaaaactgacattgaaccaccattcaccattgactctgcc

SEQ ID NO: 44 - DNA, 60 bp, Artificial
  Primer pCDF-OCPsynCterNterMIX15
tggttcaatgtcagttttttctggattgtctagtcggctgctgcccatggtatatctcct

SEQ ID NO: 45 - DNA, 46 bp, Artificial
  Primer pCDF-OCPsynCterNterNC11
aatcaggctaatcaggtgactccattcaccattgactctgcccgcg

SEQ ID NO: 46 - DNA, 45 bp, Artificial
  Primer pCDF-OCPsynCterNterNC11
agtcacctgattagcctgattgctgctgcccatggtatatctcct

SEQ ID NO: 47 - DNA, 42 bp, Artificial
  Primer pCDF-OCPsynCterNterNC9
aatcaggctaatcagccattcaccattgactctgcccgcgga

SEQ ID NO: 48 - DNA, 39 bp, Artificial
  Primer pCDF-OCPsynCterNterNC9
ctgattagcctgattgctgctgcccatggtatatctcct

SEQ ID NO: 49 - DNA, 30 bp, Artificial
  Primer pCDF-OCPsynCterNterNC7
ccattcaccattgactctgcccgcggaatt

SEQ ID NO: 50 - DNA, 48 bp, Artificial
  Primer pCDF-OCPsynCterNterNC7
agagtcaatggtgaatggagcctgattgctgctgcccatggtatatct

SEQ ID NO: 51 - DNA, 30 bp, Artificial; identical to SEQ ID NO: 49
  Primer pCDF-OCPsynCterNterNC4

SEQ ID NO: 52 - DNA, 42 bp, Artificial
  Primer pCDF-OCPsynCterNterNC4
agagtcaatggtgaatgggctgctgcccatggtatatctcct

SEQ ID NO: 53 - DNA, 42 bp, Artificial
  Primer pCDF-OCPsynCterNterC9
aaaaagagaagggctccattcaccattgactctgcccgcgga

SEQ ID NO: 54 - DNA, 39 bp, Artificial
  Primer pCDF-OCPsynCterNterC9
agcccttctctttttgctgctgcccatggtatatctcct

SEQ ID NO: 55 - DNA, 33 bp, Artificial
  Primer pCDF-OCPsynNter
catcaccatcatcaccacccattcaccattgac

SEQ ID NO: 56 - DNA, 33 bp, Artificial
  Primer pCDF-OCPsynNter
gtggtgatgatggtgatggctgctgcccatggt

SEQ ID NO: 57 - DNA, 29 bp, Artificial
  Primer OCParthroCter F pCDF-OCParthroCter NcoI/NotI
aacagaccatggcattcaccattgactcg

SEQ ID NO: 58 - DNA, 37 bp, Artificial; identical to SEQ ID NO: 32
  Primer OCParthroCter R pCDF-OCParthroCter NcoI/NotI

SEQ ID NO: 59 - DNA, 33 bp, Artificial; identical to SEQ ID NO: 55
  Primer pCDF-OCParthroNter

SEQ ID NO: 60 - DNA, 33 bp, Artificial; identical to SEQ ID NO: 56
  Primer pCDF-OCParthroNter

SEQ ID NO: 61 - DNA, 29 bp, Artificial
  Primer OCPanaCter F pCDF-OCPanaCter NcoI/NotI
aacagaccatggcaattactatcgattcc

SEQ ID NO: 62 - DNA, 34 bp, Artificial; identical to SEQ ID NO: 34
  Primer OCPanaCter R pCDF-OCPanaCter NcoI/NotI

SEQ ID NO: 63 - DNA, 36 bp, Artificial
  Primer pCDF-OCPanaNter
catcaccatcatcaccacgcaattactatcgattcc

SEQ ID NO: 64 - DNA, 33 bp, Artificial; identical to SEQ ID NO: 56
  Primer pCDF-OCPanaNter

SEQ ID NO: 65 - DNA, 47 bp, Artificial
  Primer pCDF-NC9-1123HIS
agcagcaatcaggctaatcagacttttacacaaacaaacgatcccac

SEQ ID NO: 66 - DNA, 47 bp, Artificial
  Primer pCDF-NC9-1123HIS
ctgattagcctgattgctgctgcccatggtatatctccttattaaag

SEQ ID NO: 67 - DNA, 48 bp, Artificial
  Primer pCDF-NC6-1123HIS
ggagatataccatgggcagcagcaagaggacttttacacaaacaaacg

SEQ ID NO: 68 - DNA, 32 bp, Artificial
  Primer pCDF-NC6-1123HIS
gcccatggtatatctccttattaaagttaaac

SEQ ID NO: 69 - DNA, 528 bp, Erwinia
  misc_feature: Gene sequence of Erwinia crtz gene
atgttgtggatttggaatgccctgattgttctggtcactgttatcggaatggagataacggctgcactggcgcacagatacattatgcatggctggggttggggctggcatctgtcacatcatgaaccgcataaaggctggtttgaggttaatgacctctatgccgtagtgttcgccgctctgtcgattttgctcatttatctgggcagcacgggtgtctggcccttgcagtggatcggcgcaggtatgacgctctacggcctgctctattttattgtgcatgacggcctggtgcatcagcgctggccattccgctatgttccgcgccggggttatttacgcaggctttatatggcgcaccgcatgcatcatgcggtacgaggcaaagagggctgtgtctcgtttggctttctttacgcgccgccgctgtcaaaactgcaggcgacgctgcgagaacgtcatggcgttaaacggggcgctgccagagatcagcggagcgtggagcgtgacgcgccacccgggaagtaa

SEQ ID NO: 70 - DNA, 6008 bp, Artificial
  Crt operon containing crtB, crtE, crtI and crtY genes from Erwinia uredovora under the control of the promoter of crtE
ggtaccgcacggtctgccaatccgacggaggtttatgaattttccaccttttccacaagctcaactagtattaacgatgtggatttagcaaaaaaaacctgtaaccctaaatgtaaaataacgggtaagcctgccaaccatgttatggcagattaagcgtctttttgaagggcaccgcatctttcgcgttgccgtaaatgtatccgtttataaggacagcccgaatgacggtctgcgcaaaaaaacacgttcatctcactcgcgatgctgcggagcagttactggctgatattgatcgacgccttgatcagttattgcccgtggagggagaacgggatgttgtgggtgccgcgatgcgtgaaggtgcgctggcaccgggaaaacgtattcgccccatgttgctgttgctgaccgcccgcgatctgggttgcgctgtcagccatgacggattactggatttggcctgtgcggtggaaatggtccacgcggcttcgctgatccttgacgatatgccctgcatggacgatgcgaagctgcggcgcggacgccctaccattcattctcattacggagagcatgtggcaatactggcggcggttgccttgctgagtaaagcctttggcgtaattgccgatgcagatggcctcacgccgctggcaaaaaatcgggcggtttctgaactgtcaaacgccatcggcatgcaaggattggttcagggtcagttcaaggatctgtctgaaggggataagccgcgcagcgctgaagctattttgatgacgaatcactttaaaaccagcacgctgttttgtgcctccatgcagatggcctcgattgttgcgaatgcctccagcgaagcgcgtgattgcctgcatcgtttttcacttgatcttggtcaggcatttcaactgctggacgatttgaccgatggcatgaccgacaccggtaaggatagcaatcaggacgccggtaaatcgacgctggtcaatctgttaggcccgagggcggttgaagaacgtctgagacaacatcttcagcttgccagtgagcatctctctgcggcctgccaacacgggcacgccactcaacattttattcaggcctggtttgacaaaaaactcgctgccgtcagttaaggatgctgcatgagccatttcgcggcgatcgcaccgcctttttacagccatgttcgcgcattacagaatctcgctcaggaactggtcgcgcgcggtcatcgggtgacctttattcagcaatacgatattaaacacttgatcgatagcgaaaccattggatttcattccgtcgggacagacagccatccccccggcgcgttaacgcgcgtgctacacctggcggctcatcctctggggccgtcaatgctgaagctcatcaatgaaatggcgcgcaccaccgatatgctgtgccgcgaactcccccaggcatttaacgatctggccgtcgatggcgtcattgttgatcaaatggaaccggcaggcgcgctcgttgctgaagcactgggactgccgtttatctctgtcgcctgcgcgctgcctctcaatcgtgaaccggatatgcccctggcggttatgcctttcgaatacgggaccagcgacgcggctcgcgaacgttatgccgccagtgaaaaaatttatgactggctaatgcgtcgtcatgaccgtgtcattgccgaacacagccacagaatgggcttagccccccggcaaaagcttcaccagtgtttttcgccactggcgcaaatcagccagcttgttcctgaactggattttccccgcaaagcgttaccggcttgttttcatgccgtcgggcctctgcgcgaaacgcacgcaccgtcaacgtcttcatcccgttattttacatcctcagaaaaaccccggattttcgcctcgctgggcacgcttcagggacaccgttatgggctgtttaaaacgatagtgaaagcctgtgaagaaattgacggtcagctcctgttagcccactgtggtcgtcttacggactctcagtgtgaagagctggcgcgaagccgtcatacacaggtggtggattttgccgatcagtcagccgcgctgtctcaggcgcagctggcgatcacccacggcggcatgaatacggtactggacgcgattaattaccggacgccccttttagcgcttccgctggcctttgatcagcccggcgtcgcgtcacgcatcgtttatcacggcatcggcaagcgtgcttcccgctttaccaccagccatgctttggctcgtcagatgcgttcattgctgaccaacgtcgactttcagcagcgcatggcgaaaatccagacagcccttcgtttggcagggggcaccatggccgctgccgatatcattgagcaggttatgtgcaccggtcagcctgtcttaagtgggagcggctatgcaaccgcattatgatctgattctcgtgggggctggactcgcgaatggccttatcgccctgcgtcttcagcagcagcaacctgatatgcgtattttgcttatcgacgccgcaccccaggcgggcgggaatcatacgtggtcatttcaccacgatgatttgactgagagccaacatcgttggatagctccgctggtggttcatcactggcccgactatcaggtacgctttcccacacgccgtcgtaagctgaacagcggctacttttgtattacttctcagcgtttcgctgaggttttacagcgacagtttggcccgcacttgtggatggataccgcggtcgcagaggttaatgcggaatctgttcggttgaaaaagggtcaggttatcggtgcccgcgcggtgattgacgggcggggttatgcggcaaattcagcactgagcgtgggcttccaggcgtttattggccaggaatggcgattgagccacccgcatggtttatcgtctcccattatcatggatgccacggtcgatcagcaaaatggttatcgcttcgtgtacagcctgccgctctcgccgaccagattgttaattgaagacacgcactatattgataatgcgacattagatcctgaatgcgcgcggcaaaatatttgcgactatgccgcgcaacagggttggcagcttcagacactgctgcgagaagaacagggcgccttacccattactctgtcgggcaatgccgacgcattctggcagcagcgccccctggcctgtagtggattacgtgccggtctgttccatcctaccaccggctattcactgccgctggcggttgccgtggccgaccgcctgagtgcacttgatgtctttacgtcggcctcaattcaccatgccattacgcattttgcccgcgagcgctggcagcagcagggctttttccgcatgctgaatcgcatgctgtttttagccggacccgccgattcacgctggcgggttatgcagcgtttttatggtttacctgaagatttaattgcccgtttttatgcgggaaaactcacgctgaccgatcggctacgtattctgagcggcaagccgcctgttccggtattagcagcattgcaagccattatgacgactcatcgttaaagagcgactacatgaaaccaactacggtaattggtgcaggcttcggtggcctggcactggcaattcgtctacaagctgcggggatccccgtcttactgcttgaacaacgtgataaacccggcggtcgggcttatgtctacgaggatcaggggtttacctttgatgcaggcccgacggttatcaccgatcccagtgccattgaagaactgtttgcactggcaggaaaacagttaaaagagtatgtcgaactgctgccggttacgccgttttaccgcctgtgttgggagtcagggaaggtctttaattacgataacgatcaaacccggctcgaagcgcagattcagcagtttaatccccgcgatgtcgaaggttatcgtcagtttctggactattcacgcgcggtgtttaaagaaggctatctaaagctcggtactgtcccttttttatcgttcagagacatgcttcgcgccgcacctcaactggcgaaactgcaggcatggagaagcgtttacagtaaggttgccagttacatcgaagatgaacatctgcgccaggcgttttctttccactcgctgttggtgggcggcaatcccttcgccacctcatccatttatacgttgatacacgcgctggagcgtgagtggggcgtctggtttccgcgtggcggcaccggcgcattagttcaggggatgataaagctgtttcaggatctgggtggcgaagtcgtgttaaacgccagagtcagccatatggaaacgacaggaaacaagattgaagccgtgcatttagaggacggtcgcaggttcctgacgcaagccgtcgcgtcaaatgcagatgtggttcatacctatcgcgacctgttaagccagcaccctgccgcggttaagcagtccaacaaactgcagactaagcgcatgagtaactctctgtttgtgctctattttggtttgaatcaccatcatgatcagctcgcgcatcacacggtttgtttcggcccgcgttaccgcgagctgattgacgaaatttttaatcatgatggcctcgcagaggacttctcactttatctgcacgcgccctgtgtcacggattcgtcactggcgcctgaaggttgcggcagttactatgtgttggcgccggtgccgcatttaggcaccgcgaacctcgactggacggttgaggggccaaaactacgcgaccgtatttttgcgtaccttgagcagcattacatgcctggcttacggagtcagctggtcacgcaccggatgtttacgccgtttgattttcgcgaccagcttaatgcctatcatggctcagccttttctgtggagcccgttcttacccagagcgcctggtttcggccgcataaccgcgataaaaccattactaatctctacctggtcggcgcaggcacgcatcccggcgcaggcattcctggcgtcatcggctcggcaaaagcgacagcaggtttgatgctggaggatctgatatgaataatccgtcgttactcaatcatgcggtcgaaacgatggcagttggctcgaaaagttttgcgacagcctcaaagttatttgatgcaaaaacccggcgcagcgtactgatgctctacgcctggtgccgccattgtgacgatgttattgacgatcagacgctgggctttcaggcccggcagcctgccttacaaacgcccgaacaacgtctgatgcaacttgagatgaaaacgcgccaggcctatgcaggatcgcagatgcacgaaccggcgtttgcggcttttcaggaagtggctatggctcatgatatcgccccggcttacgcgtttgatcatctggaaggcttcgccatggatgtacgcgaagcgcaatacagccaactggatgatacgctgcgctattgctatcacgttgcaggcgttgtcggcttgatgatggcgcaaatcatgggcgtgcgggataacgccacgctggaccgcgcctgtgaccttgggctggcatttcagttgaccaatattgctcgcgatattgtggacgatgcgcatgcgggccgctgttatctgccggcaagctggctggagcatgaaggtctgaacaaagagaattatgcggcacctgaaaaccgtcaggcgctgagccgtatcgcccgtcgtttggtgcaggaagcagaaccttactatttgtctgccacagccggcctggcagggttgcccctgcgttccgcctgggcaatcgctacggcgaagcaggtttaccggaaaataggtgtcaaagttgaacaggccggtcagcaagcctgggatcagcggcagtcaacgaccacgcccgaaaaattaacgctgctgctggccgcctctggtcaggcccttacttcccggatgcgggctcatcctccccgccctgcgcatctctggcagcgcccgctctagcgccatgtctttcccggagcgt

SEQ ID NO: 71 - DNA, 45 bp, Artificial
  forward primer Crt-pBAD
aacagagtttaaaccggtaccgcacggtctgccaatccgacggag

SEQ ID NO: 72 - DNA, 42 bp, Artificial
  reverse primer Crt-pBAD
gccgtttctagaacgctccgggaaagacatggcgctagagcg

SEQ ID NO: 73 - DNA, 38 bp, Artificial
  forward primer T7lacOCP-pBADfull
aacagaacatgtgccataccgcgaaaggttttgcgcca

SEQ ID NO: 74 - DNA, 38 bp, Artificial
  reverse primer T7lacOCP-pBADfull
aacagaacatgtcaagacccgtttagaggccccaaggg

SEQ ID NO: 75 - DNA, 6369 bp, Artificial
  Crt operon of Erwinia herbicola containing the crtE, crtY, crtI and crtB genes in the pAC-BETA plasmid
agatctaaaggcacagcgtctcatgcttcgcacaatgtaaaactgcttcagaacctggcgagagctatccgcgcggtctacggttaactgatactaaaagacaattcagcgggtaaccttgcaatggtgagtggcagtaaagcgggcgtttcgcctcatcgcgaaatagaagtaatgagacaatccattgacgatcacctggctggcctgttacctgaaaccgacagccaggatatcgtcagccttgcgatgcgtgaaggcgtcatggcacccggtaaacggatccgtccgctgctgatgctgctggccgcccgcgacctccgctaccagggcagtatgcctacgctgctcgatctcgcctgcgccgttgaactgacccataccgcgtcgctgatgctcgacgacatgccctgcatggacaacgccgagctgcgccgcggtcagcccactacccacaaaaaatttggtgagagcgtggcgatccttgcctccgttgggctgctctctaaagcctttggtctgatcgccgccaccggcgatctgccgggggagaggcgtgcccaggcggtcaacgagctctctaccgccgtgggcgtgcagggcctggtactggggcagtttcgcgatcttaacgatgccgccctcgaccgtacccctgacgctatcctcagcaccaaccacctcaagaccggcattctgttcagcgcgatgctgcagatcgtcgccattgcttccgcctcgtcgccgagcacgcgagagacgctgcacgccttcgccctcgacttcggccaggcgtttcaactgctggacgatctgcgtgacgatcacccggaaaccggtaaagatcgcaataaggacgcgggaaaatcgacgctggtcaaccggctgggcgcagacgcggcccggcaaaagctgcgcgagcatattgattccgccgacaaacacctcacttttgcctgtccgcagggcggcgccatccgacagtttatgcatctgtggtttggccatcaccttgccgactggtcaccggtcatgaaaatcgcctgataccgcccttttgggttcaagcagtacataacgatggaaccacattacaggagtagtgatgaatgaaggacgagcgccttgttcagcgtaagaacgatcatctggatatcgttctcgacccccgtcgcgccgtaactcaggctagcgcaggttttgagcgctggcgctttacccactgcgccctgccagagctgaattttagcgacatcacgctggaaaccaccttcctgaatcggcagctacaggctccgctgctgatcagctccatgaccggcggcgttgagcgctcgcgccatatcaaccgccacctcgccgaggcggcgcaggtgctaaaaattgcgatgggggtgggctcccagcgcgtcgccattgagagcgacgcgggcttagggctggataaaaccctgcggcagctggctccggacgtgccgctgctggcgaacctcggcgcggcgcagctgaccggcagaaaaggtattgattacgcccgacgggccgtggagatgatcgaggcggatgcgctgattgtgcacctaaacccgctgcaggaggcgctacagcccggcggcgatcgcgactggcgcggacggctggcggctattgaaactctggtccgcgagctgcccgttccgctggtggtgaaagaggtgggagccggtatctcccgaaccgtggccgggcagctgatcgatgccggcgttaccgtgattgacgtcgcgggcgcgggcggcaccagctgggccgccgttgaaggcgagcgggcggccaccgagcagcagcgcagcgtggccaacgtctttgccgactgggggatccccaccgctgaggcgctggttgacattgccgaggcctggccgcagatgccccttattgcctcgggcgggattaaaaacggctcgaccagccgcgctacgttgccgaggctaatctggtgatcacccacggcggtctcaataccgtactggatgcgctggctgccgcgacgccggtgctggcggtgccactctctttcgaccagcccgccgtggctgcccggctggtctataacgggctgggtcgccgggtatcgcgctttgccagacagcagacgctggcggatgagattgcccaactgctgggggatgagacgctgcatcagcgtctggcgacggcccgccagcagcttaacgacgccgggggcacgccccgtgcggcgaccctgattgaacaggccatagcagggagtgagagcgtatcgtgagggatctgattttagtcggcggcggcctggccaacgggctgatcgcctggcgtctgcgccagcgctacccgcagcttaacctgctgctgatcgaggccggggagcagcccggcgggaaccatacctggtcattccatgaagacgatctgactcccgggcagcacgcctggctggccccgctggtggcccacgcctggccgggctatgaggtgcagtttcccgatcttcgccgtcgcctcgcgcgcggctactactccattacctcagagcgctttgccgaggccctgcatcaggcgctgggggagaacatctggctaaactgttcggtgagcgaggtgttacccaatagcgtgcgccttgccaacggtgaggcgctgcttgccggagcggtgattgacggacgcggcgtgaccgccagttcggcgatgcaaaccggctatcagctctttcttggtcagcagtggcggctgacacagccccacggcctgaccgtaccgatcctgatggatgccacggtggcgcagcagcagggctatcgctttgtctacacgctgccgctctccgccgacacgctgctgatcgaggatacgcgctacgccaatgtcccgcagcgtgatgataatgccctacgccagacggttaccgactatgctcacagcaaagggtggcagctggcccagcttgaacgcgaggagaccggctgtctgccgattaccctggcgggtgacatccaggctctgtgggccgatgcgccgggcgtgccgcgctcgggaatgcgggctgggctatttcaccctaccactggctattcgctgccgctggcggtggcccttgccgacgcgattgccgacagcccgcggctgggcagcgttccgctctatcagctcacccggcagtttgccgaacgccactggcgcaggcagggattcttccgcctgctgaaccggatgcttttcctggccgggcgcgaggagaaccgctggcgggtgatgcagcgcttttatgggctgccggagcccaccgtagagcgcttttacgccggtcggctctctctctttgataaggcccgcattttgacgggcaagccaccggttccgctgggcgaagcctggcgggcggcgctgaaccattttcctgacagacgagataaaggatgaaaaaaaccgttgtgattggcgcaggctttggtggcctggcgctggcgattcgcctgcaggcggcagggatcccaaccgtactgctggagcagcgggacaagcccggcggtcgggcctacgtctggcatgaccagggctttacctttgacgccgggccgacggtgatcaccgatcctaccgcgcttgaggcgctgttcaccctggccggcaggcgcatggaggattacgtcaggctgctgccggtaaaacccttctaccgactctgctgggagtccgggaagaccctcgactatgctaacgacagcgccgagcttgaggcgcagattacccagttcaacccccgcgacgtcgagggctaccggcgctttctggcttactcccaggcggtattccaggagggatatttgcgcctcggcagcgtgccgttcctctcttttcgcgacatgctgcgcgccgggccgcagctgcttaagctccaggcgtggcagagcgtctaccagtcggtttcgcgctttattgaggatgagcatctgcggcaggccttctcgttccactccctgctggtaggcggcaaccccttcaccacctcgtccatctacaccctgatccacgcccttgagcgggagtggggggtctggttccctgagggcggcaccggggcgctggtgaacggcatggtgaagctgtttaccgatctgggcggggagatcgaactcaacgcccgggtcgaagagctggtggtggccgataaccgcgtaagccaggtccggctggcggatggtcggatctttgacaccgacgccgtagcctcgaacgctgacgtggtgaacacctataaaaagctgctcggccaccatccggtggggcagaagcgggcggcagcgctggagcgcaagagcatgagcaactcgctgtttgtgctctacttcggcctgaaccagcctcattcccagctggcgcaccataccatctgttttggtccccgctaccgggagctgatcgacgagatctttaccggcagcgcgctggcggatgacttctcgctctacctgcactcgccctgcgtgaccgatccctcgctcgcgcctcccggctgcgccagcttctacgtgctggccccggtgccgcatcttggcaacgcgccgctggactgggcgcaggaggggccgaagctgcgcgaccgcatctttgactaccttgaagagcgctatatgcccggcctgcgtagccagctggtgacccagcggatctttaccccggcagacttccacgacacgctggatgcgcatctgggatcggccttctccatcgagccgctgctgacccaaagcgcctggttccgcccgcacaaccgcgacagcgacattgccaacctctacctggtgggcgcaggtactcaccctggggcgggcattcctggcgtagtggcctcggcgaaagccaccgccagcctgatgattgaggatctgcaatgagccaaccgccgctgcttgaccacgccacgcagaccatggccaacggctcgaaaagttttgccaccgctgcgaagctgttcgacccggccacccgccgtagcgtgctgatgctctacacctggtgccgccactgcgatgacgtcattgacgaccagacccacggcttcgccagcgaggccgcggcggaggaggaggccacccagcgcctggcccggctgcgcacgctgaccctggcggcgtttgaaggggccgagatgcaggatccggccttcgctgcctttcaggaggtggcgctgacccacggtattacgccccgcatggcgctcgatcacctcgacggctttgcgatggacgtggctcagacccgctatgtcacctttgaggatacgctgcgctactgctatcacgtggcgggcgtggtgggtctgatgatggccagggtgatgggcgtgcgggatgagcgggtgctggatcgcgcctgcgatctggggctggccttccagctgacgaatatcgcccgggatattattgacgatgcggctattgaccgctgctatctgcccgccgagtggctgcaggatgccgggctgaccccggagaactatgccgcgcgggagaatcgggccgcgctggcgcgggtggcggagcggcttattgatgccgcagagccgtactacatctcctcccaggccgggctacacgatctgccgccgcgctgcgcctgggcgatcgccaccgcccgcagcgtctaccgggagatcggtattaaggtaaaagcggcgggaggcagcgcctgggatcgccgccagcacaccagcaaaggtgaaaaaattgccatgctgatggcggcaccggggcaggttattcgggcgaagacgacgagggtgacgccgcgtccggccggtctttggcagcgtcccgtttaggcgggcggccatgacgttcacgcaggatcgcctgtaggtcggcaggcttgcgggcgtaaataaaaccgaaggagacgcagccctcccggccgcgcaccgcgtggtgcaggcggtgggcgacgtagagccgcttcaggtagccccggcgcgggatcccgcctcgccgtgctgtccggtctcaacctgatccgccagaatcgagccaacgggatcggccagcacgaattcggtatgcggagagtgtttggcaaaccatgcctgcaagccacccagcgtgccgccggagccaacgcctactaccacggcatcaaccctgcctgccagctggtcgaacagctccggtgcggtggtggtggcgtgcgccagcgggttggccgggttagagaactgatcaatatagtaagcacccggcgtctcctctgccaggcggcgggcgtagtcctgatagtactccgggtggccctttgtcacgtcggagcgggtcaggcgcacatcaacacccagcgcacgcaggtggtagatct